(19) United States
(12) Patent Application Publication
(10) Pub. No.: US 2015/0122646 A1
(43) Pub. Date: May 7, 2015

(54) Mediator-less Electrochemical Glucose Sensing Procedure Employing the Leach-proof Covalent Binding of an Enzyme(s) to Electrodes and Products Thereof

(71) Applicants: NATIONAL UNIVERSITY OF SINGAPORE, Singapore (SG); KING SAUD UNIVERSITY, Riyadh (SA)

(72) Inventors: Khalid Ali Al-Rubeaan, Riyadh (SA); Dan Zheng, Singapore (SG); Fwu-Shan Sheu, Singapore (SG); Sandeep Kumar Vashist, Singapore (SG)

(21) Appl. No.: 14/398,669

(22) PCT Filed: May 3, 2013
(86) PCT No.: PCT/SG2013/000175

§ 371 Date: Nov. 3, 2014

Related U.S. Application Data
(60) Provisional application No. 61/641,886, filed on May 3, 2012.

(51) Int. Cl. G01N 27/327; C12Q 1/00

(52) U.S. Cl. CPC G01N 27/3272; C12Q 1/003
USPC 204/403.14; 435/177; 435/175

(57) ABSTRACT

The present disclosure generally relates to devices and procedures for the development of glucose oxidase-bound electrodes by a covalent binding of glucose oxidase on amine-functionalized electrodes. More particularly, the present disclosure is related to covalently-bound enzyme-coated electrodes that are leach-proof and highly stable for continuous glucose monitoring. The glucose oxidase-bound electrodes are employed for the development of a mediator-less electrochemical glucose sensing procedure having no interference from biological substances and drugs.

### CROSS REFERENCE TO RELATED APPLICATIONS

This application claims priority to and is a non-provisional conversion of U.S. Provisional Patent Application Ser. No. 61/641,886, entitled “A Mediator-less Electrochemical Glucose Sensing Procedure Employing the Leach-proof Covalent Binding of Enzyme to the Electrodes and Products Thereof” filed on 3 May 2012, which is incorporated herein by reference in its entirety.

### TECHNICAL FIELD

This disclosure generally relates to devices and procedures for the development of glucose oxidase-bound electrodes by a covalent binding of glucose oxidase on amine-functionalized electrodes. More particularly, the covalently-bound enzyme-coated electrodes were leach-proof and highly stable for continuous glucose monitoring. The glucose oxidase-bound electrodes are employed for the development of a mediator-less electrochemical glucose sensing procedure having no interference with biological substances and drugs.

The disclosure also relates- to the development of a highly-simplified procedure for producing stable and leach-proof glucose oxidase-bound electrodes for mediator-less electrochemical detection of glucose. The developed technology is applicable to a highly stable continuous glucose monitoring (CGM) system, glucose meter or closed-loop system for diabetic monitoring. In an embodiment, the said developed glucose sensing strategy employing the devised enzyme-bound electrodes can be applied to or form a portion of a continuous glucose monitoring system (CGMS).

The disclosure further relates to the development of a bienzyme-based mediator-less electrochemical (EC) glucose sensing technology and sensing procedure. The developed glucose sensing technology has a wide dynamic range and increased sensitivity. The developed glucose sensing technology is applicable to blood glucose monitoring devices (BGMD's), i.e., a continuous glucose monitoring system (CGMS), glucose meter or closed-loop system. The use of two enzymes, i.e., glucose oxidase (GOx) and horseradish peroxidase (HRP), eliminates the oxygen limitation for the detection of glucose, which increases the dynamic range for glucose sensing.

### BACKGROUND

Diabetes has become a global epidemic and is a major concern for all nations. The annual cost of diabetes management, which was 11.2% of the total global healthcare expenditure, is an unbearable economic burden. The disease is increasing at an alarming rate. The monitoring of blood glucose in diabetics is therefore the most predominant diagnostic test with over 16.7 billion tests per year and an annual market of USD 6.1 billion (http://www.researchandmarkets.com/reports/338842). The market is currently served mainly by large industries such as Abbott, Bayer, Roche, LifeScan, Dexcom and Minimed. A diabetic has to daily monitor his/her glucose level in frequent intervals to keep the glucose level within the physiological range in order to avoid diabetic complications. There are a number of problems with glucose measurements that the inventors propose to address. The first is the limited durability of measurement cartridges typically to be used one time after purchase. Secondly, the use of a specific electron mediator in the measurement system can cause potentially fatal errors in glucose measurements as was seen in certain commercial products. This was followed by immediate recalls of these commercial products by the industry after the US FDA's public health notification in 2009 (http://www.fda.gov/MedicalDevices/Safety/AlertsandNotices/PublicHealthNotifications/ucm176992.htm).

However, mediators are still employed in glucose meters and several reports confirm interferences from physiological substances and medications (Diabetic Medicine, DOI: 10.1111/j. 1464-5491.2011.03362.x; Am. J. Clin. Pathol. 113, 75-86, 2000; more ref. s). Similarly, the use of a positive applied potential such as 0.4 V versus the reference electrodes in many glucose meters can introduce similar errors in glucose estimation because many physiological substances and drugs have a redox potential between 300 to 700 mV.

Various technologies have been used for the detection of glucose. These include the electrochemical techniques that are currently employed by almost all industries for manufacturing the BGMD. However, several other glucose detection concepts, such as those based on non-invasive glucose monitoring, (Diab. Res. Clin. Prac. 77, 16-40, 2007) have also been demonstrated. The GlucoWatch Biographer of Cygnus, USA, which was an FDA approved system based on the transdermal extraction of interstitial fluid by reverse iontophoresis, was immediately withdrawn due to its non-acceptance by the market due to inaccuracy, false alarm, skin irritation, sweating and long warm up time. Similarly, Diasensor from BICO Inc. and Pendra from Pendragon Medical Ltd. were also withdrawn from the market due to serious concerns about their accuracy. Despite significant research efforts, a reliable non-invasive BGMD is not available.

The non-enzymatic detection of glucose has also been demonstrated by many approaches using nanomaterials including the highly cited results of PI's group (Electrochemistry Communications 6, 66-70, 2004; Electroanalysis 17, 89-96, 2005; Nanotechnology 17, 2334-2339, 2006; Analytica Chimica Acta 594,175-183, 2007), which however fails in terms of selectivity, reliability to test the entire pathophysiological range of glucose concentration in blood serum, and reproducibility in bioanalytical procedures.

### SUMMARY

In accordance with an aspect of the present disclosure, a device includes an electrochemical (EC) glucose biosensor, which is mediator-less and employs a negative applied potential vs. a reference electrode, which makes the device free of physiological interferences in glucose detection, sensing, measurement, and/or reading including with respect to medications taken by patients. More particularly, the use of a secondary substrate such as graphene or multi-walled carbon nanotubes (MWCNTs) can obviate the need of a mediator as graphene and MWCNTs have excellent electrical conductivity and can act as an electron wire to facilitate direct electron transfer between the redox center of the enzyme, glucose oxidase, and the electrode's surface. A secondary substrate such as graphene or other secondary substrate on the electrode provides electrochemical signal enhancement due to its large surface area, which increases the sensitivity of glucose detection.

In accordance with related aspects of the present disclosure, a bio-analytical procedure for the preparation of covalently-bound leach-proof glucose oxidase-coated electrodes and a mediator-less electrochemical glucose sensing strategy using an applied potential of −450 mV for continuous glucose monitoring are disclosed. More particularly, the developed technology enables glucose detection in the human patho-physiological range, i.e., 0.5-32 mM, without any biofouling of the disclosed electrode or interference from physiological substances/drugs. There is no significant decrease in the glucose sensing signal when the same electrode is employed continuously for the detection of a particular glucose concentration for at least 4 weeks. Therefore, the devised strategy would be potentially useful for the development of a continuous glucose monitoring system (CGMS).

In accordance with a further aspect of the present disclosure, the developed technology described herein has overcome problems of the existing technologies that have been addressed in our recent comprehensive review, i.e., “Technology behind commercial devices for blood glucose monitoring in diabetic management: A review” in Analytica Chimica Acta (2011), volume 703, pp. 124-136 incorporated herein by reference in its entirety.

In accordance with yet another aspect of the present disclosure, the said developed technology is not only useful for developing CGMS devices but also utilizes a generic strategy that can be employed for preparing covalently-bound enzyme-coated electrodes for the electrochemical detection of other analytes. Therefore, the said developed technology can be employed for the development of enzyme-based electrochemical sensors.

The developed mediator-less electrochemical glucose sensing technology has immense potential for the development of CGMS based on its wider dynamic range, use of a negative applied potential and absence of potential interferences from physiologically interfering substances. The various modifications of the developed strategy have also been used for devising several strategies for glucose detection. Moreover, as previously described, the developed strategy utilizes a generic strategy that can be employed for preparing covalently-bound enzyme-coated electrodes for the electrochemical detection of other analytes.

In addition or as an alternative to the foregoing, embodiments of the present disclosure can be modified strategies employing various nanomaterials such as graphene nano platelets (GNPs), multiwalled carbon nanotubes (MWCNTs) and poly-L-lysine (PLL) as secondary substrates. The strategy can work on many different types of nanomaterials. Therefore, various nanocomposites can be made and used for electrochemical glucose sensing.

A highly-simplified procedure has been developed, which enables the preparation of highly stable and leach-proof glucose-oxidase bound electrodes. The developed enzyme-bound electrodes have a wide dynamic range of 0.5-48 mM without any decrease in the glucose sensing signal for about four weeks when stored at room temperature under ambient conditions. There is no evidence of biofouling even after storage in blood samples for five days. Moreover, the electrochemical strategy employed for glucose detection using the developed-enzyme-bound electrodes was mediator-less and used −450 mV as the applied potential. Therefore, there was no interference with the physiological substances, which is a key concern for the development of commercial blood glucose meters. The developed procedure for preparing enzyme-bound electrodes and the developed electrochemical glucose sensing strategy are ideal for the development of a CGMS, glucose meter or closed-loop system for diabetic monitoring as they can be easily transduced or translated to practice in industrial and clinical settings. The developed simplified procedure is appropriate for the commercial mass production of enzyme-bound electrodes, employing techniques such as screen-printing.

A bienzyme-based mediator-less EC glucose sensing procedure has also been developed, which has a wide dynamic range and increased sensitivity for glucose detection. The use of HRP with GOx eliminates the oxygen limitation in EC glucose sensing as it reduces the hydrogen peroxide, produced by the conversion of glucose to gluconolactone, back to water and oxygen. Moreover, the decreased hydrogen peroxide will significantly enhance the resistance to biofouling in the enzyme-coated electrodes prepared by the developed technology. The absence of a mediator and the use of a negative applied potential (−450 mV) versus the Ag/AgCl reference electrode makes the developed glucose sensing procedure less prone to interference with physiological substances and medications. The various strategies developed employing the bienzyme-based mediator-less EC glucose sensing procedure have a wide dynamic range that covers the clinically-relevant patho-physiological range in diabetics, i.e., 0.5-28 mM glucose. Therefore, the developed bienzyme technology has tremendous potential for the development of a CGMS, glucose meter or closed-loop system for diabetic monitoring. The developed bioanalytical procedure is simple and can be easily transduced or translated to practice for the commercial mass-production of enzyme-bound electrodes in industries employing simple techniques such as screen-printing.

A first aspect of the present disclosure provides a mediator-less biosensor for detecting an analyte within a detection environment, the mediator-less biosensor comprising:

a substrate having an electrically conductive chemically modified surface to which a functionalizing agent is covalently bonded; and

a first enzyme immobilized relative to the surface by way of covalent bonding to one of the functionalizing agent, a polymer chemically bonded to the functionalizing agent, and a nano-engineered material chemically bonded to the functionalizing agent,

wherein the mediator-less biosensor is configured for direct electron transfer between the analyte and the first enzyme in response to application of a negative electrical potential to the surface relative to the detection environment.

In embodiments, the mediator-less biosensor described above maintains a substantially stable analyte detection capability for a period of approximately 20 days.

In embodiments, the electrically conductive chemically modified surface carries hydroxyl groups to which the functionalizing agent is covalently bound.

In embodiments, the functionalizing agent comprises an organofunctional alkoxysilane compound. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 4% by volume. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 2% by volume. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 1% by volume.

In embodiments, the polymer comprises an amine functional polymer. In embodiments, the polymer comprises one of an amino acid polymer (e.g., poly-l-lysine) and a glucosamine based polymer (e.g., chitosan).

In embodiments, the nano-engineered material includes at least one of graphene nano-platelets, multi-walled carbon nanotubes, and nanocrystalline cellulose.

In embodiments, the mediator-less biosensor described above further comprises a selective diffusion membrane that limits exposure of the first enzyme to substances within the detection environment.

In embodiments, the substrate carries one of a metal (e.g., platinum, gold) and a carbon based material. In embodiments, the substrate comprises a glassy carbon electrode.

In embodiments, the direct electron transfer between the analyte and one of the first biomolecule and the first enzyme is substantially unaffected by the presence of molecular substances other than the analyte including biological species and drug metabolites.

In embodiments, the first enzyme is suitable for detecting one of glucose, cholesterol, alcohol, lactate, acetylcholine, choline, hypoxanthine, and xanthine. In embodiments, the first enzyme comprises glucose oxidase.

In embodiments, the mediator-less biosensor described above is capable of detecting glucose across substantially the entire diabetic pathological concentration range. In embodiments, the mediator-less biosensor is capable of detecting glucose across a concentration range of approximately 0.5-32 mM.

In embodiments, the first enzyme becomes covalently bonded to the surface by way of exposure of the surface to the functionalizing agent thereby creating a functionalized surface, followed by exposure of the functionalized surface to the first enzyme.

In embodiments, the first enzyme becomes covalently bonded to the surface by way of exposing the surface to a fluid medium carrying a mixture of the first enzyme and the functionalizing agent.

In embodiments, the polymer becomes covalently bonded to the surface by way of exposure of the surface to a suspension comprising the polymer and the functionalizing agent.

In embodiments, the polymer becomes covalently bonded to the surface and the first enzyme becomes covalently bonded to the polymer by way of exposure of the surface to a fluid medium comprising the functionalizing agent, the polymer, and the first enzyme.

In embodiments, the nano-engineered material becomes covalently bonded to the surface by way of exposure of the surface to the functionalizing agent thereby creating a functionalized surface, followed by exposure of the functionalized surface to a polar dispersion agent carrying the nano-engineered material.

In embodiments, the nano-engineered material becomes covalently bonded to the surface by way of exposure of the surface to a suspension comprising the nano-engineered material and the functionalizing agent.

In embodiments, the nano-engineered material becomes covalently bonded to the surface and the first enzyme becomes covalently bonded to the nano-engineered material by way of exposure of the surface to a fluid medium comprising the functionalizing agent, the nano-engineered material, and the first enzyme.

In embodiments, the mediator-less biosensor described above further comprises a second enzyme immobilized relative to the surface by way of covalent bonding to one of the functionalizing agent, the polymer, and the nano-engineered material.

In embodiments, the second enzyme can reduce a byproduct of an electrochemical analyte detection reaction. In embodiments, the second enzyme comprises horseradish peroxidase. In embodiments, the second enzyme increases at least one of dynamic analyte detection range and analyte detection sensitivity.

In embodiments, the first enzyme comprises glucose oxidase and the mediator-less biosensor is capable of detecting glucose across a concentration range of approximately 0.5-48 mM.

In embodiments, the first enzyme and the second enzyme become covalently bonded to the functionalizing agent by way of exposure of the surface to a fluid medium carrying the functionalizing agent, the first enzyme, and the second enzyme.

In embodiments, the first enzyme and the second enzyme become covalently bonded to the polymer by way of exposure of the surface to a fluid medium carrying the functionalizing agent, the polymer, the first enzyme, and the second enzyme.

In embodiments, the first enzyme and the second enzyme become covalently bonded to the nano-engineered material by way of exposure of the surface to a fluid medium carrying the functionalizing agent, the nano-engineered material, the first enzyme, and the second enzyme.

A second aspect of the present disclosure provides a method for manufacturing a mediator-less biosensor configured for detecting an analyte in a detection environment, the method comprising:

providing a substrate having an electrically conductive chemically modified surface; covalently bonding a functionalizing agent to the surface; and

performing an immobilization process comprising one of:

(a) covalently bonding a first enzyme to the functionalizing agent;

(b) covalently bonding a polymer to the functionalizing agent and covalently bonding the first enzyme to the polymer; and

(c) covalently bonding a nano-engineered material to the functionalizing agent and covalently bonding the first enzyme to the nano-engineered material,

wherein the mediator-less biosensor is configured for direct electron transfer between the analyte and the first enzyme in response to application of a negative electrical potential to the surface relative to the detection environment.

In embodiments, the step of providing a substrate having an electrically conductive chemically modified surface comprises:

providing a substrate having an electrically conductive surface; and

exposing the surface to a surface modification agent such that the surface carries hydroxyl groups.

In embodiments, the functionalizing agent comprises an organofunctional alkoxysilane compound. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 4% by volume. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 2% by volume. In embodiments, the functionalizing agent comprises an organofunctional alkoxysilane compound, wherein the functionalizing agent becomes covalently bound to the surface by way of exposure of the surface to a fluid medium in which the functionalizing agent is present at a concentration of less than approximately 1% by volume.

In embodiments, the polymer comprises an amine functional polymer. In embodiments, the polymer comprises one of an amino acid polymer and a glucosamine based polymer.

In embodiments, the nano-engineered material includes at least one of graphene nano-platelets, multi-walled carbon nanotubes, and nanocrystalline cellulose.

In embodiments, the method for manufacturing a mediator-less biosensor configured for detecting an analyte in a detection environment described above further comprises a selective diffusion membrane that limits exposure of the first enzyme to substances within the detection environment.

In embodiments, the substrate carries one of a metal and a carbon based material. In embodiments, the substrate comprises a glassy carbon electrode.

In embodiments, the direct electron transfer between the analyte and the first enzyme is substantially unaffected by the presence of molecular substances other than the analyte including biological species and drug metabolites.

In embodiments, the first enzyme is suitable for detecting one of glucose, cholesterol, alcohol, lactate, acetylcholine, choline, hypoxanthine, and xanthine. In embodiments, the first enzyme comprises glucose oxidase.

In embodiments, the mediator-less biosensor described above is capable of detecting glucose across substantially the entire diabetic pathological concentration range.

In embodiments, the mediator-less biosensor described above is capable of detecting glucose across a concentration range of approximately 0.5-32 mM.

In embodiments, the first enzyme becomes covalently bonded to the surface by way of exposure of the surface to the functionalizing agent thereby creating a functionalized surface, followed by exposure of the functionalized surface to the first enzyme.

In embodiments, the first enzyme becomes covalently bonded to the surface by way of exposing the surface to a fluid medium carrying a mixture of the first enzyme and the functionalizing agent.

In embodiments, the polymer becomes covalently bonded to the surface by way of exposure of the surface to a suspension comprising the polymer and the functionalizing agent.

In embodiments, the polymer becomes covalently bonded to the surface and the first enzyme becomes covalently bonded to the polymer by way of exposure of the surface to a fluid medium comprising the functionalizing agent, the polymer, and the first enzyme.

In embodiments, the nano-engineered material becomes covalently bonded to the surface by way of exposure of the surface to the functionalizing agent thereby creating a functionalized surface, followed by exposure of the functionalized surface to a polar dispersion agent carrying the nano-engineered material.

In embodiments, the nano-engineered material becomes covalently bonded to the surface by way of exposure of the surface to a suspension comprising the nano-engineered material and the functionalizing agent.

In embodiments, the nano-engineered material becomes covalently bonded to the surface and the first enzyme becomes covalently bonded to the nano-engineered material by way of exposure of the surface to a fluid medium comprising the functionalizing agent, the nano-engineered material, and the first enzyme.

In embodiments, the immobilization process involves the first enzyme and a second enzyme different than the first enzyme, and wherein the immobilization process comprises one of:

(a) covalently bonding the first enzyme and the second enzyme to the functionalizing agent;

(b) covalently bonding a polymer to the functionalizing agent and covalently bonding the first enzyme and the second enzyme to the polymer; and

(c) covalently bonding a nano-engineered material to the functionalizing agent and covalently bonding the first enzyme and the second enzyme to the nano-engineered material.

In embodiments, the second enzyme can reduce a byproduct of an electrochemical analyte detection reaction. In embodiments, the second enzyme comprises horseradish peroxidase. In embodiments, the second enzyme increases at least one of dynamic analyte detection range and analyte detection sensitivity.

In embodiments, the first enzyme comprises glucose oxidase, wherein the mediator-less biosensor is capable of detecting glucose across a concentration range of approximately 0.5-48 mM.

In embodiments, the first enzyme and the second enzyme become covalently bonded to the functionalizing agent by way of exposure of the surface to a fluid medium carrying the functionalizing agent, the first enzyme, and the second enzyme.

In embodiments, the first enzyme and the second enzyme become covalently bonded to the polymer by way of exposure of the surface to a fluid medium carrying the functionalizing agent, the polymer, the first enzyme, and the second enzyme.

In embodiments, the first enzyme and the second enzyme become covalently bonded to the nano-engineered material by way of exposure of the surface to a fluid medium carrying the functionalizing agent, the nano-engineered material, the first enzyme, and the second enzyme.

A third aspect of the present disclosure provides a mediator-less enzyme-coated electrode comprising:

a. a primary substrate; and

b. an enzyme covalently bound to said primary substrate.

In embodiments, the mediator-less enzyme-coated electrode described above further comprises a selective diffusion membrane.

A fourth aspect of the present disclosure provides a mediator-less enzyme-coated electrode comprising:

a. a primary substrate;

b. a secondary substrate; and

c. an enzyme covalently bound to said primary and secondary substrates.

A fifth aspect of the present disclosure provides a method of preparing a mediator-less enzyme-coated electrode comprising:

a. covalently binding an enzyme to at least a primary substrate to form a mediator-less enzyme coated electrode.

A sixth aspect of the present disclosure provides a method of electrochemically detecting an analyte in a sample in the absence of a mediator, comprising:

a. exposing a mediator-less enzyme-coated electrode to a sample comprising an analyte;

b. applying a negative potential to said mediator-less enzyme-coated electrode; and

c. detecting said analyte in said sample.

A seventh aspect of the present disclosure provides a stable enzyme-coated electrode comprising:

a. a primary substrate;

b. an enzyme attached to said primary substrate; and

c. a selective diffusion membrane,

wherein said electrode maintains a stable analyte sensing signal for at least about 20 days.

In embodiments, the stable enzyme-coated electrode described above further comprises a secondary substrate.

In embodiments, said electrode has a dynamic range of about 0.5 mM to about 48 mM.

An eighth aspect of the present disclosure provides a method for reducing byproducts of an electrochemical analyte detection reaction comprising:

a. providing a mediator-less enzyme-coated electrode comprising at least two enzymes;

b. applying a negative potential to said mediator-less enzyme-coated electrode;

c. exposing said mediator-less enzyme-coated electrode having a negative applied potential to a sample containing an analyte to initiate an electrochemical analyte detection reaction; and

d. detecting the level of said analyte in the sample,

wherein at least one of said at least two enzymes catalyzes the reduction of a byproduct of said electrochemical analyte detection reaction.

A ninth aspect of the present disclosure provides a method for increasing the sensitivity of an enzyme-coated electrode in the absence of a mediator comprising:

a. providing an enzyme-coated electrode comprising at least two enzymes, wherein at least one of said at least two enzymes comprises a catalase;

b. applying a negative potential to said enzyme-coated electrode;

c. exposing said enzyme-coated electrode at a negative applied potential to a sample containing an analyte, in the absence of a mediator, to initiate an electrochemical analyte detection reaction; and

d. catalyzing a reduction of byproducts of said electrochemical analyte detection reaction with said catalase, in the absence of a mediator, thereby increasing the sensitivity of said enzyme-coated electrode to said analyte.

### BRIEF DESCRIPTION OF THE DRAWINGS

1) Multi-Step EC Glu Sensing

FIG. 1A: is a schematic diagram of the developed procedure for the development of covalently-bound enzyme-coated electrodes. Route 1: passive adsorption based strategy, as described in 1.1.3 and Route 2: covalent binding based strategy, as described in 1.1.2.

FIG. 1B: is an effect of 3-Aminopropyltriethoxysilane (APTES) concentration on the electrochemical detection of glucose using Nafion/GOx/APTES/GCE1.

FIG. 1C: shows electrochemical glucose sensing assay curves for the detection of glucose.

FIG. 1D: shows electrochemical glucose sensing assay curves for the detection of Streck artificial blood glucose standards.

FIG. 1E: shows an effect of interfering substances on the developed electrochemical glucose sensing strategy.

FIG. 1F: shows a continuous detection of 4 mM glucose employing the developed covalently-bound enzyme-coated electrode.

FIG. 1G: shows an effect of biofouling on the electrochemical glucose sensing of the developed covalently-bound enzyme-coated electrode.

FIG. 1H: shows a production reproducibility of the developed direct GOx based strategy.

FIG. 2A: is a schematic representation of the developed graphene nano platelets (GNPs) based strategy for electrochemical glucose sensing.

FIG. 2B: shows an effect of APTES concentration on the electrochemical detection of glucose using the developed GNPs based strategy.

FIG. 2C: is a comparison of assay curves for the electrochemical detection of glucose using Nafion/GOx-EDC activated/GNPs-APTES/GCE and Nafion/GOx/APTES/GCE.

FIG. 2D: shows a glucose sensing curve for detection of Streck artificial blood glucose.

FIG. 2E: shows an effect of interfering substances on the developed GNPs based strategy.

FIG. 2F: shows a production reproducibility of the developed GNPs based strategy.

FIG. 2G: shows a stability of the developed GNPs based glucose sensor at room temperature (RT) in a dry state.

FIG. 2H: shows a BCA protein assay for the determination of GOx binding to developed glucose sensors that were used for glucose detection for 9 weeks.

FIG. 2I: shows a determination of the effect of biofouling by keeping the sensor immersed in 1 mM Sugar-Chex blood glucose linearity standard for 7 days but used intermittently each day for detecting 6.8 mM Sugar-Chex blood glucose linearity standard in triplicate.

FIG. 3A: is a schematic representation of the developed poly-L-lysine (PLL) based electrochemical glucose sensing strategies.

FIG. 3B: shows an assay curve for the electrochemical detection of glucose using Nafion/GOx-EDC activated/PLL-APTES/GCE.

FIG. 3C: shows an electrochemical detection of glucose using Streck artificial blood glucose.

FIG. 3D: shows an effect of interfering substances on the electrochemical detection of glucose using Nafion/GOx-EDC activated/PLL-APTES/GCE.

FIG. 3E: shows the production reproducibility of the developed PLL based strategy.

FIG. 4A: is a schematic representation of the developed multiwalled carbon nanotubes (MWCNTs) based strategies for electrochemical glucose sensing.

FIG. 4B: shows an effect of varying APTES concentrations on MWCNT (dispersed in DMF).

FIG. 4C: shows an effect of varying APTES concentrations on MWCNT (dispersed in APTES)-based electrochemical glucose biosensing formats.

FIG. 4D: shows an overlay plot of various formats based on the optimized APTES concentration for a particular format.

FIG. 4E: shows a use of a MWCNT (dispersed in DMF) based electrochemical glucose biosensing format for the detection of various Streck blood glucose linearity standards.

FIG. 4F: shows an effect of physiological interferences and medications on the specific detection of glucose.

FIG. 4G: shows the production reproducibility for the development of 25 GOx-functionalized GCEs based on the detection of 4 mM glucose.

2) EC Glu Sensing

FIG. 5A: is a schematic representation of the developed highly-simplified procedure for the development of enzyme-bound electrodes.

FIG. 5B: shows an assay curve for the electrochemical detection of glucose using the developed enzyme-bound electrodes.

FIG. 5C: shows an assay curve for the electrochemical detection of glucose in Streck artificial blood glucose standards using the developed enzyme-bound electrodes.

FIG. 5D: shows an effect of interfering substances on the developed electrochemical glucose sensing strategy.

FIG. 5E: shows a reproducibility of the developed simplified procedure for preparing GOx-bound glassy carbon electrodes (GCE), which was demonstrated by the electrochemical detection of 8 mM glucose using 25 freshly prepared GOx-bound GCEs.

FIG. 5F: shows a stability of the developed GOx-bound GCE in terms of the electrochemical detection of 8 mM glucose, when stored at RT in dry state.

FIG. 5G: shows a stability of the developed GOx-bound GCE in terms of the electrochemical detection of 8 mM glucose, when stored at RT in 50 mM PBS, pH 7.4.

FIG. 5H: shows a stability of the developed GOx-bound GCE in terms of the electrochemical detection of 8 mM glucose, when stored at 4° C. in dry state.

FIG. 5I: shows a stability of the developed GOx-bound GCE in terms of the electrochemical detection of 8 mM glucose, when stored at 4° C. in 50 mM PBS, pH 7.4.

FIG. 5J: shows an effect of biofouling on the electrochemical glucose sensing of developed electrodes, which was demonstrated by storing the developed GOx-bound electrodes in Streck's Sugar-Chex blood glucose linearity standard for many days. No biofouling was observed on the developed electrodes.

FIG. 5K: shows a BCA protein assay based determination of the amount of GOx bound when the developed strategy was employed on different substrates to demonstrate its generic multisubstrate-compatible nature.

FIG. 6A: shows a schematic representation of a modified developed simplified procedure for preparing GOx-bound electrodes employing graphene nano platelets (GNPs) as an additional intermediate substance or secondary substrate.

FIG. 6B: shows an assay curve for the electrochemical detection of glucose using a developed highly-simplified preparation procedure.

FIG. 6C: shows an assay curve for Streck blood glucose linearity standards.

FIG. 6D: shows an effect of interfering substances on the developed electrochemical glucose sensing strategy using a highly-simplified preparation procedure.

FIG. 6E: shows a reproducibility of the developed simplified procedure for preparing GOx-bound GNPs-coated GCE, which was demonstrated by the electrochemical detection of 8 mM glucose using 25 freshly prepared GNPs-GOx-bound GCEs.

FIG. 7A: is a schematic representation of a modified developed simplified procedure for preparing GOx-bound electrodes employing poly-L-lysine (PLL) as an additional intermediate substance or secondary substrate.

FIG. 7B: shows an assay curve for the electrochemical detection of glucose using the Nafion/PLL-GOx/GCE.

FIG. 7C: shows an assay curve for the electrochemical detection of glucose in Streck artificial blood glucose standards using the Nafion/PLL-GOx/GCE.

FIG. 7D: shows an effect of interfering substances on the developed PLL-based glucose sensing strategy.

FIG. 8A: is a schematic representation of a modified developed simplified procedure for preparing GOx-bound electrodes employing multiwalled carbon nanotubes (MWCNTs) as an additional intermediate substance or secondary substrate.

FIG. 8B: shows an assay curve for the electrochemical detection of glucose using the Nafion/APTES-MWCNTs-GOx/GCE.

FIG. 8C: shows an assay curve for the electrochemical detection of glucose in Streck artificial blood glucose standards using the Nafion/APTES-MWCNTs-GOx/GCE.

FIG. 8D: shows an effect of interfering substances on the developed MWCNTs based glucose sensing strategy.

3) 1-Step Bienzyme EC Glu Sensing

FIG. 9A: is a schematic representation of the developed bienzyme-based mediator-less EC sensing procedure, where GOx and HRP are bound to amine-functionalized GCE and then covered with Nafion.

FIG. 9B: shows an assay curve for the electrochemical glucose detection using the developed strategy.

FIG. 9C: shows an effect of interfering substances on the EC glucose detection by the developed strategy.

FIG. 10A: is a schematic representation of the developed bienzyme-based mediator-less EC sensing procedure employing graphene nano platelets (GNPs).

FIG. 10B: shows an assay curve for the electrochemical glucose detection employing the developed strategy.

FIG. 10C: shows an effect of interfering substances on the EC glucose detection by the developed strategy.

FIG. 11A: is a schematic representation of the developed bienzyme-based mediator-less EC sensing procedure employing poly-L-lysine (PLL).

FIG. 11B: shows an assay curve for the electrochemical glucose detection using the developed strategy.

FIG. 11C: shows an effect of interfering substances on the EC glucose detection by the developed strategy.

FIG. 12A: is a schematic representation of the developed bienzyme-based mediator-less EC sensing procedure employing multi-walled carbon nanotubes (MWCNTs).

FIG. 12B: shows an assay curve for the electrochemical glucose detection using the developed strategy.

FIG. 12C: shows an effect of interfering substances on the EC glucose detection by the developed strategy.

FIG. 13A: is a schematic representation of the developed bienzyme-based mediator-less EC sensing procedure employing chitosan (CS).

FIG. 13B: shows an assay curve for the electrochemical glucose detection using the developed strategy.

FIG. 13C: shows an effect of interfering substances on the EC glucose sensing by the developed strategy.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the term “about”, in the context of concentrations of components, conditions, other measurement values, etc., means +/−5% of the stated value, or +/−4% of the stated value, or +/−3% of the stated value, or +/−2% of the stated value, or +/−1% of the stated value, or +/−0.5% of the stated value, or +/−0% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Various embodiments in accordance with the present disclosure are directed to mediator-less electrochemical analyte (e.g., glucose) sensing devices and procedures. With respect to particular embodiments directed to the immobilization of enzymes such as glucose oxidase relative to an electrode structure, some of such embodiments include the following:
(1) Development of glucose oxidase-bound electrodes by glucose oxidase immobilization relative to an amine-functionalized electrode, such as by way of covalent bonding of glucose oxidase with respect to or on a surface corresponding to an amine-functionalized electrode.
(2) A highly-simplified procedure for the preparation of highly stable and leach-proof glucose-oxidase bound electrodes.
(3) Bienzyme-based mediator-less electrochemical glucose sensing.

Development of Mediator-Less Electrochemical Glucose Sensing Procedures and Devices.

Experiments to develop mediator-less electrochemical glucose sensing procedures and devices in accordance with the embodiments of the present disclosure:

### Experiments 1

### A Development of Glucose Oxidase-Bound Electrodes by a Covalent Binding of Glucose Oxidase on Amine-Functionalized Electrode

Table 1 shows a comparison of the developed covalent glucose sensing strategies with various leading commercial glucose meters.

| | | | | LifeScan | | | | |
|---|---|---|---|---|---|---|---|---|
| | Abbott | Bayer | Roche Accu- | one | | Graphene | | |
| Analytical | Freestyle | Contour | Chek | Touch | | Nano | Poly-L- | |
| Parameters | Lite | USB | Advantage | Ultra 2 | GOx | Platelets | lysine | MWCNTs |
| Dynamic | 2-28 | 2-28 | 2-28 | 2-28 | 0.5-32 | 0.5-32 | 0.5-16 | 0.5-32 |
| range | Problems | Problems | Problems | Problems | | | | |
| | >22 & <2 | >24 & <2 | >22 & <2 | >22 & | | | | |
| | | | | <2 | | | | |
| Assay time | 5 s | 5 s | 5 s | 5 s | 4 s | 4 s | 4 s | 4 s |
| Mediator | Os complex | Ferricyanide | Ferricyanide | Ferricyanide | None | None | None | None |
| added | | | | | | | | |
| Enzymes | GDH | GDH | GDH | GOx | GOx- | GOx-HRP | GOx- | GOx-HRP |
| | | | | | HRP | | HRP | |
| Precision | <5% | <5% | <5% | <5% | <5% | <5% | <5% | <5% |
| Interferences | No | N.M. | 30 μg/mL | 30 μg/mL | No | No | No | No |
| | | | Ascorbic | Ascorbic | | | | |
| | | | acid, 20 μg/mL | acid | | | | |
| | | | Acetaminophen, | | | | | |
| | | | 40 μg/mL | | | | | |
| | | | Dopamine | | | | | |
| Drugs | N.M. | N.M. | N.M. | N.M. | No | No | No | No |
| Potential | −0.16 V | 0.4 V | 0.4 V | 0.4 V | −0.45 V | −0.45 V | −0.45 V | −0.45 V |
| Multisubstrate- | No | No | No | No | Yes | Yes | Yes | Yes |
| compatible | | | | | | | | |

Table 2 shows a comparison of the developed covalent glucose sensing strategies with various leading commercial continuous glucose monitoring systems.

| | Noninvasive: | | | | | | | |
|---|---|---|---|---|---|---|---|---|
| | GlucoWatch | Subcutaneous: | Subcutaneous: | | | | | |
| | G2 | Freestyle | Guardian | | | | | |
| | Biographer | Navigator | REAL-Time | Subcutaneous: | | Graphene | | |
| Analytical | (Johnson & | (Abbott | (Medtronic | DexCom | | Nano | Poly-L- | |
| Parameters | Johnson) | Diabetes) | MiniMed) | STS CGMS | GOx | Platelets | lysine | MWCNTs |
| Dynamic | 2-22 | 2-22 | 2-22 | 2-22 | 0.5-32 | 0.5-32 | 0.5-16 | 0.5-32 |
| range | Problems | Problems | Problems | Problems | | | | |
| | >22 & <2 | >24 & <2 | >22 & <2 | >22 & <2 | | | | |
| Assay time | N.M. | N.M. | N.M. | N.M. | 4 s | 4 s | 4 s | 4 s |
| Mediator | None | Os | None | None | None | None | None | None |
| added | | complex | | | | | | |
| Enzymes | GOx | GOx | GOx | GOx | GOx- | GOx-HRP | GOx- | GOx-HRP |
| | | | | | HRP | | HRP | |
| Precision | <5% | <5% | <5% | <5% | <5% | <5% | <5% | <5% |
| Interferences | N.M. | No | N.M. | N.M. | No | No | No | No |
| Drugs | N.M. | N.M. | N.M. | N.M. | No | No | No | No |
| Potential | 0.42 V | −0.2 V | N.M. | N.M. | −0.45 V | −0.45 V | −0.45 V | −0.45 V |
| Multisubstrate- | No | No | No | No | Yes | Yes | Yes | Yes |
| compatible | | | | | | | | |

Preparation of Covalently-Bound Leach-Proof Glucose Oxidase-Coated Electrodes and a Mediator-Less Electrochemical Glucose Sensing Strategy

Particular procedures to prepare covalently-bound leach-proof glucose oxidase-coated electrodes and a mediator-less electrochemical glucose sensing strategy in accordance with embodiments of the present disclosure are provided hereafter.

Materials and Equipment Used

Material and equipment used in this experiment are shown in Table 3.

TABLE 3

| Description | Company | Cat. No. |
|---|---|---|
| 3-Aminopropyltriethoxysilane | Sigma | A3648 |
| Glucose oxidase | Sigma | G7141 |
| D-glucose | Sigma | G7528 |
| 70 wt. % Glutaraldehyde | Sigma | G7776 |
| 5 wt. % Nafion | Sigma | 527084 |
| N,N-Dimethylformamide | Sigma | D4551 |
| Ascorbic acid | Sigma | A5960 |
| Uric acid | Sigma | U2625 |
| Acetaminophen | Sigma | A7085 |
| Dopamine hydrochloride | Sigma | H8502 |
| Creatinine | Sigma | C4255 |
| Bilirubin | Sigma | B4126 |
| Salicylic acid | Sigma | 247588 |
| Tetracycline | Sigma | 268054 |
| Bilirubin | Sigma | B4126 |
| Ibuprofen | Sigma | I4883 |
| Tolazamide | Sigma | T2408 |
| Tolbutamide | Sigma | T0891 |
| (+)-Ephedrin-hydrochloride | Sigma | 857335 |
| Hydrochloric acid | Sigma | 320331 |
| Sodium hydroxide | Sigma | S8045 |
| Acetone | Sigma | 179124 |
| Ethanol | Sigma | 459844 |
| Poly-L-lysine | Sigma | P8920 |
| BupH phosphate buffered saline | Thermo Scientific | 28372 |
| Block ™ BSA (10% in PBS) | Thermo Scientific | 37525 |
| BupH MES buffered saline | Thermo Scientific | 28390 |
| 1-Ethy-(3-dimethylaminopropyl) | Thermo Scientific | 22981 |
| carbodiimide•HCl | | |
| Sugar-Chex Linearity, Low 12345 | Streck, Inc. | 290311 |
| | (USA) | |
| Multi-walled carbon nanotubes | NanoLab, Inc. | PD15LL1-5 |
| | (USA) | |
| Graphene Nano Platelets | CheapTubes. | Grade 2 |
| | Inc. (USA) | |
| Glassy carbon working electrode | CH Instruments, | CHI104 |
| | Inc (USA) | |
| Platinum wire counter electrode | CH Instruments, | CHI115 |
| | Inc | |
| Silver/silver chloride reference | CH Instruments, | CHI111 |
| electrode | Inc | |
| CHI660A electrochemical work- | CH Instruments, | CHI660A |
| station | Inc | |
| BASi C3 Cell Stand | BASi | EF-1085 |
| EDP3-plus electronic pipettes with | Rainin | E3-20 |
| LTS, 2-20 μL | | |
| EDP3-plus electronic pipettes with | Rainin | E3-200 |
| LTS, 20-200 μL | | |
| EDP3-plus electronic pipettes with | Rainin | E3-1000 |
| LTS, 100-1000 μL | | |
| Ultrasonic cleaner (Model: 2510) | Branson | B2510E-MT |
| | | CPN-952-236 |
| Eppendorf microtubes | Sigma | Z 606340 |
| Water purification system (Model: | Millipore | ZRQSVP0UK |
| Direct Q) | | |
| SigmaPlot software | Systat | version 11.2 |

50 mM PBS was employed for making GOx and glucose dilutions and for washing after the process steps of the developed procedure. GOx stock solution, prepared by mixing equal volumes of 10 mg mL⁻¹ GOx and 5% glutaraldehyde, was stored at 4° C. and used for experiments after equilibrating for 30 min at RT.

1.1 Procedures or Methods for Directing GOx Binding

1.1.1 Surface Cleaning and Generation of Hydroxyl Groups on GCE Primary Substrate

Glassy carbon electrodes (GCE, 3 mm diameter, CH Instruments, Austin, Tex., USA) were polished consecutively using 0.3 and 0.05 μm alumina powder, and subsequently cleaned by putting in an ultrasonic bath (Model 2510, Branson) for 20 min. GCEs were then dipped in 1% KOH for 5 mM to generate hydroxyl groups on their surface.

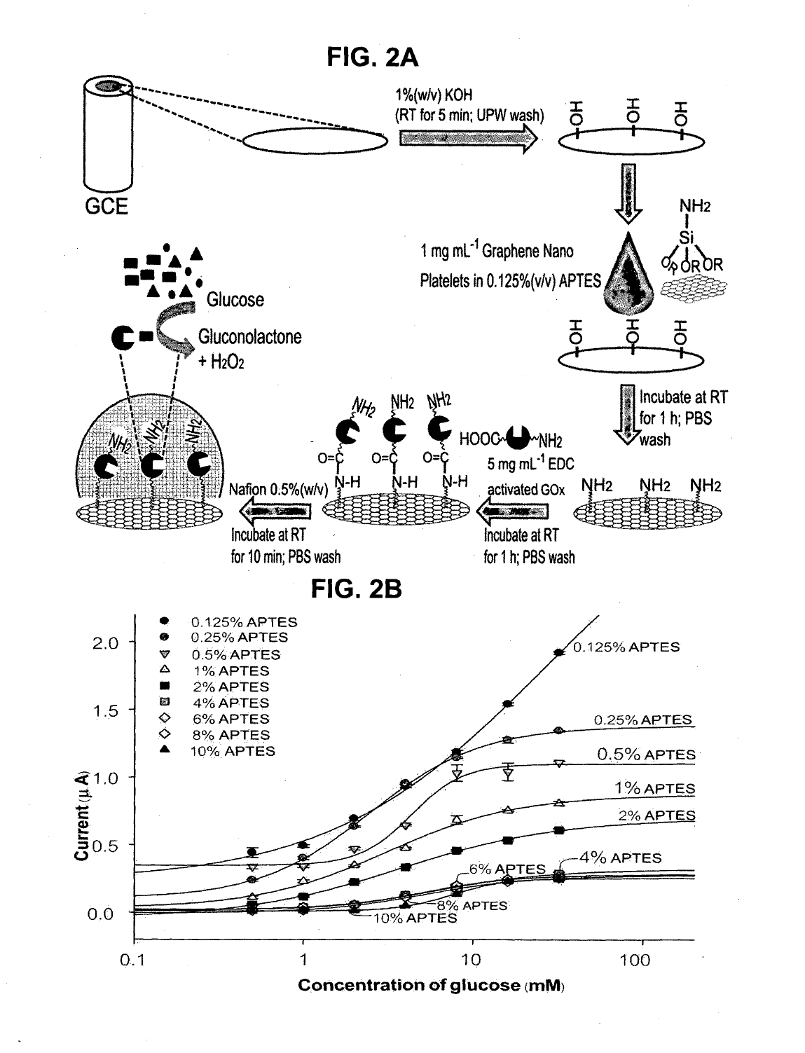

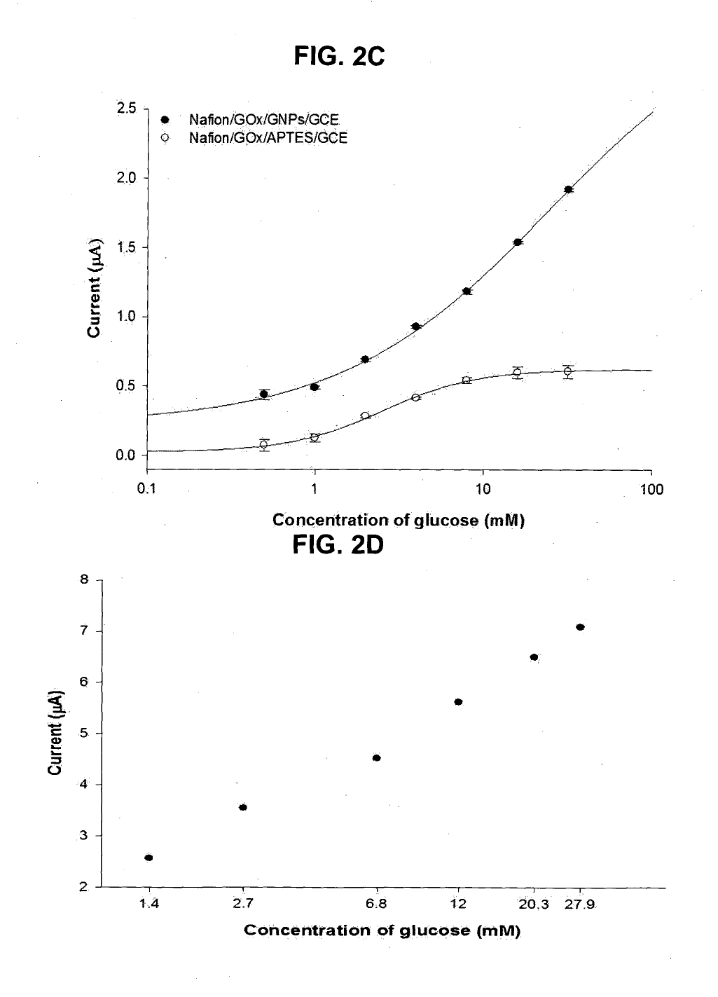

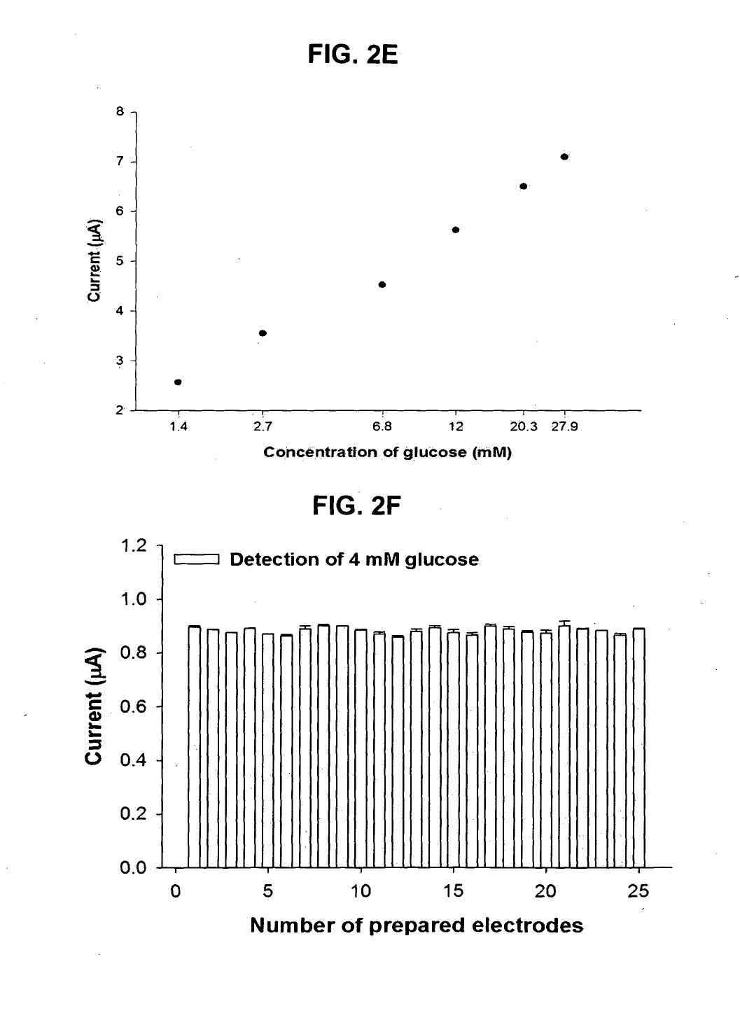

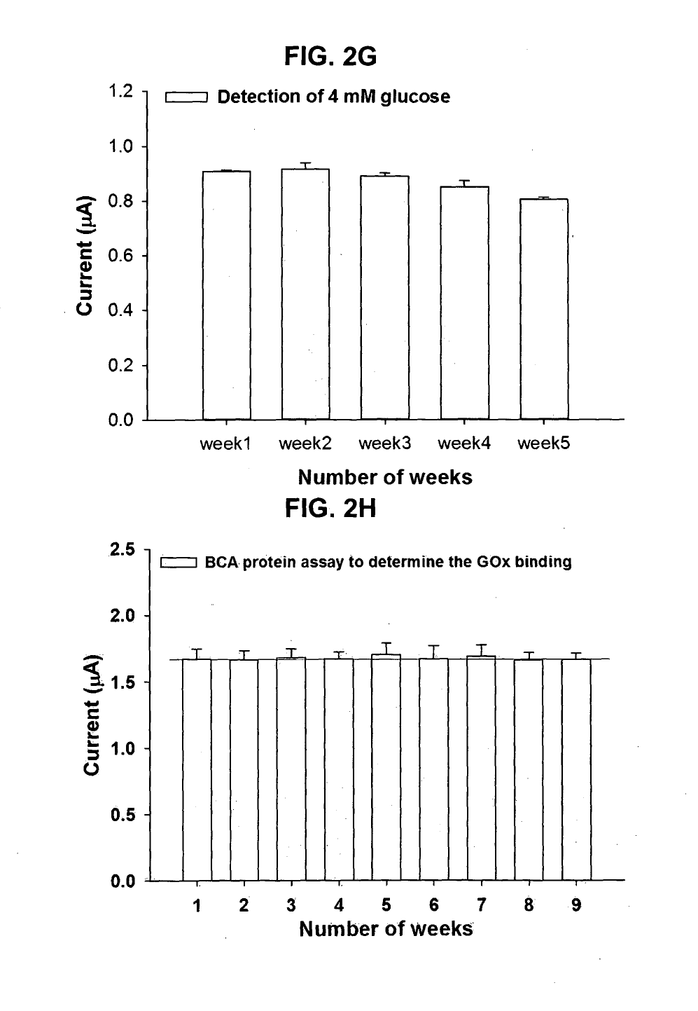

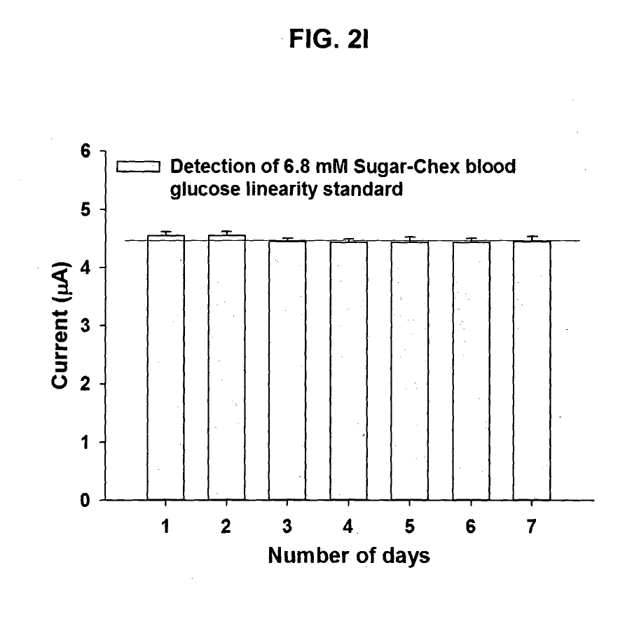

1.1.2 Developed Covalent Strategy Employing 1-Ethy-(3-dimethylaminopropyl) carbodiimide (EDC) Based Cross-Linking (Effect of APTES Concentration Shown in FIG. 2)

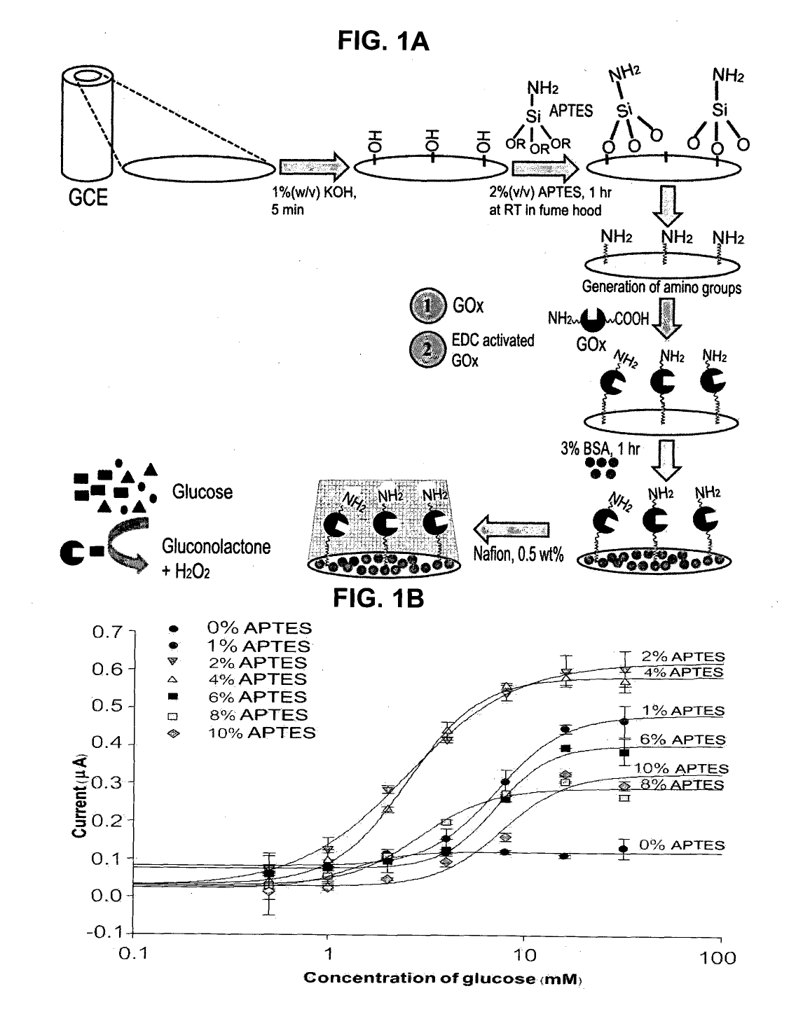

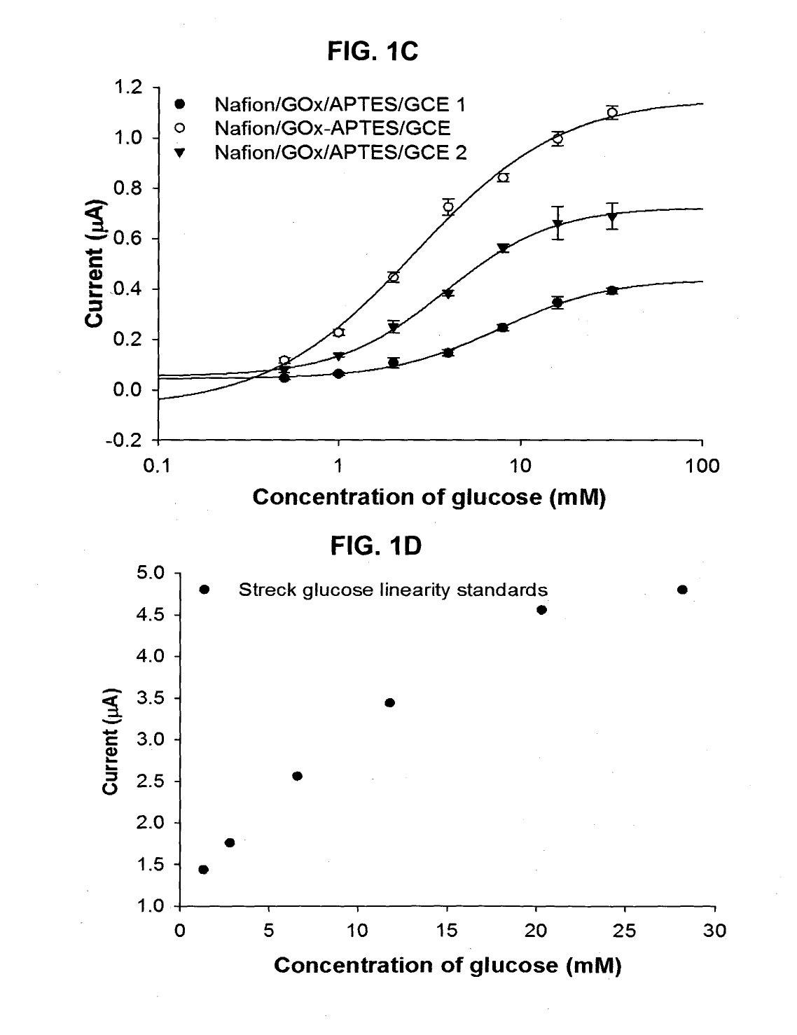

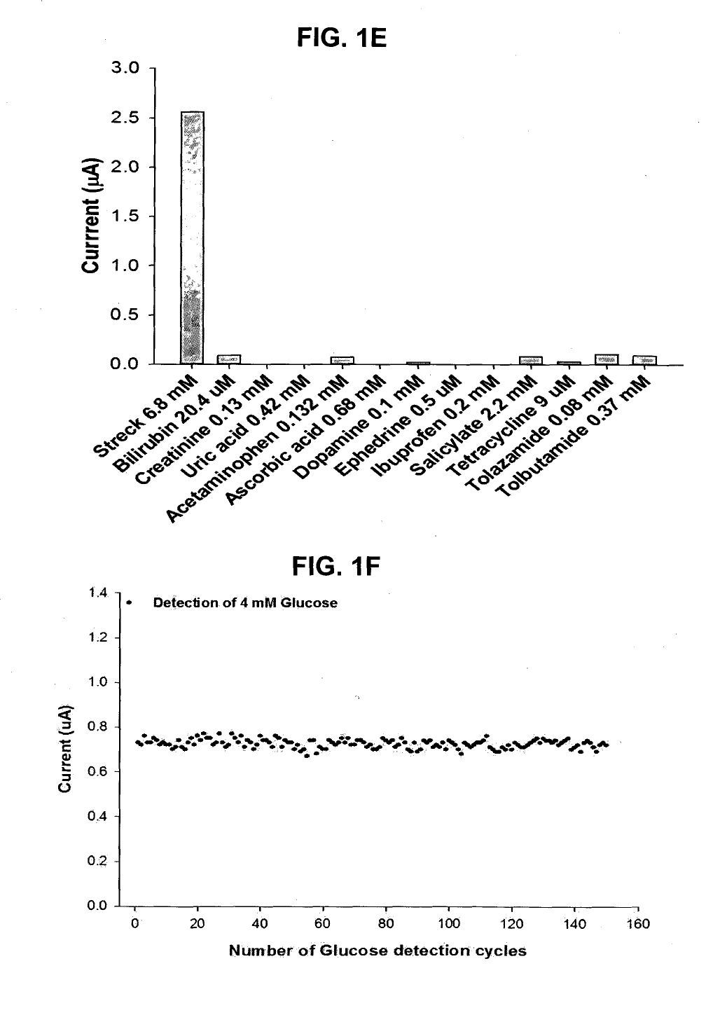

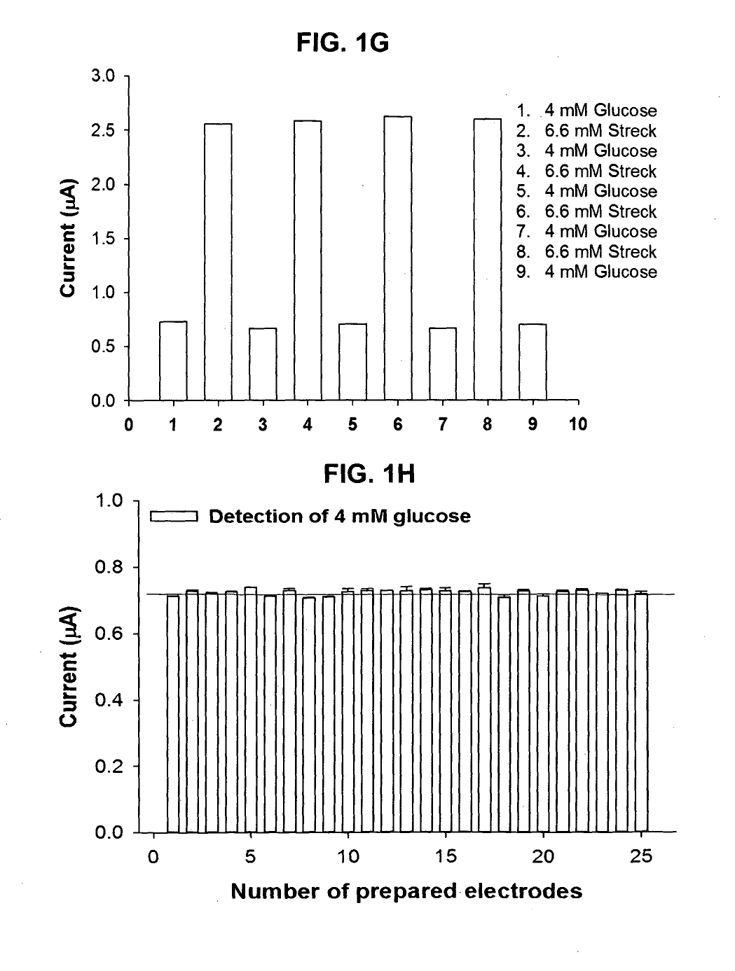

As described in a route 2 of FIG. 1, 3 μL of 2% APTES was drop-casted on GCE and dried at RT for 1 h. The APTES-functionalized GCE electrode was then washed thoroughly with ultrapure water to form APTES/GCE. 30 μL of 5 mg mL⁻¹ GOx solution was mixed with 2 μL of 0.12 g mL⁻¹ EDC solution for 15 min at RT to form EDC-GOx cross-linking solution. 4 μL of this EDC-GOx cross-linking solution was then drop-casted on APTES/GCE and dried at RT for 1 h. Thereafter, these modified electrodes were thoroughly washed with PBS to form GOx-APTES/GCE. The non-specific binding sites on the electrode were then blocked by drop-casting 4 μL of 3% BSA solution on GOx-APTES/GCE and dried at RT for 1 h followed by washing thoroughly with 50 mM PBS to form blocked GOx-APTES/GCE. Finally, 3 μL of 0.5% Nafion was drop-casted and dried at RT to form Nafion/GOx-APTES/GCE followed by extensive washing with 50 mM PBS. Nafion acts as the glucose-limiting membrane, which allows diffusion of the glucose molecules but prevents the diffusion of contaminating substances and interferences.

1.1.3 Passive Strategy and Control Electrode

As described in a route 1 of FIG. 1, two passive strategies were employed for comparison with the developed covalent crosslinking strategy. In the first strategy, GOx was directly drop-casted on APTES/GCE and dried at RT for 1 h followed by washing thoroughly with PBS to form a GOx/APTES/GCE. The subsequent steps were similar to the covalent strategy and led to the formation of Nafion/GOx/APTES/GCE 1. The second strategy employed a procedure very similar to the first one with the exception that there were no washings between the steps. The formed electrode was denoted as Nafion/GOx/APTES/GCE 2. The first control experiment, where GCE was not modified by APTES before the immobilization of GOx on GCE, led to the formation of Nafion/GOx/GCE. Whereas in the second control experiment, APTES/GCE was blocked by BSA before the immobilisation of GOx, thereby leading to the formation of Nafion/GOx/BSA/APTES/GCE.

1.1.4. Electrochemical Analysis

All electrochemical measurements were done at RT on CHI 660A electrochemical workstation using a three electrode system, i.e., developed working electrode, Pt counter electrode and 3 M Ag/AgCl reference electrode. The amperometric response of glucose was recorded in stirred PBS at −450 mV vs. Ag/AgCl.

1.1.5. Detection of Glucose

(i) Assay Curve for Glucose and Streck Sugar-Chex Blood Glucose Linearity Standards

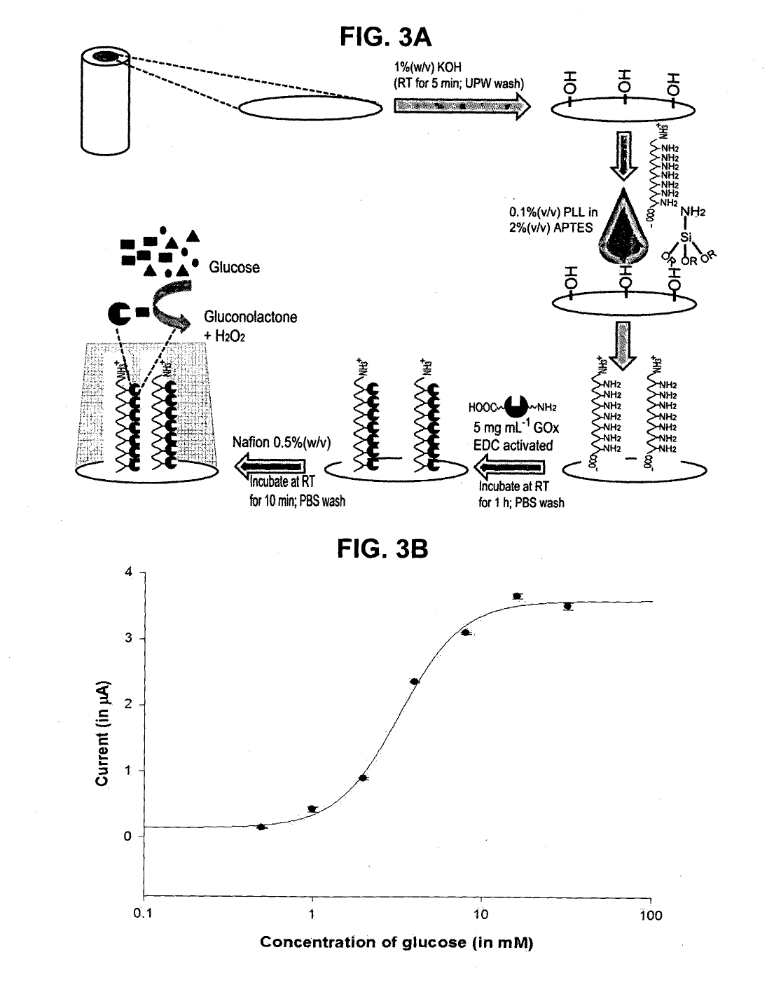

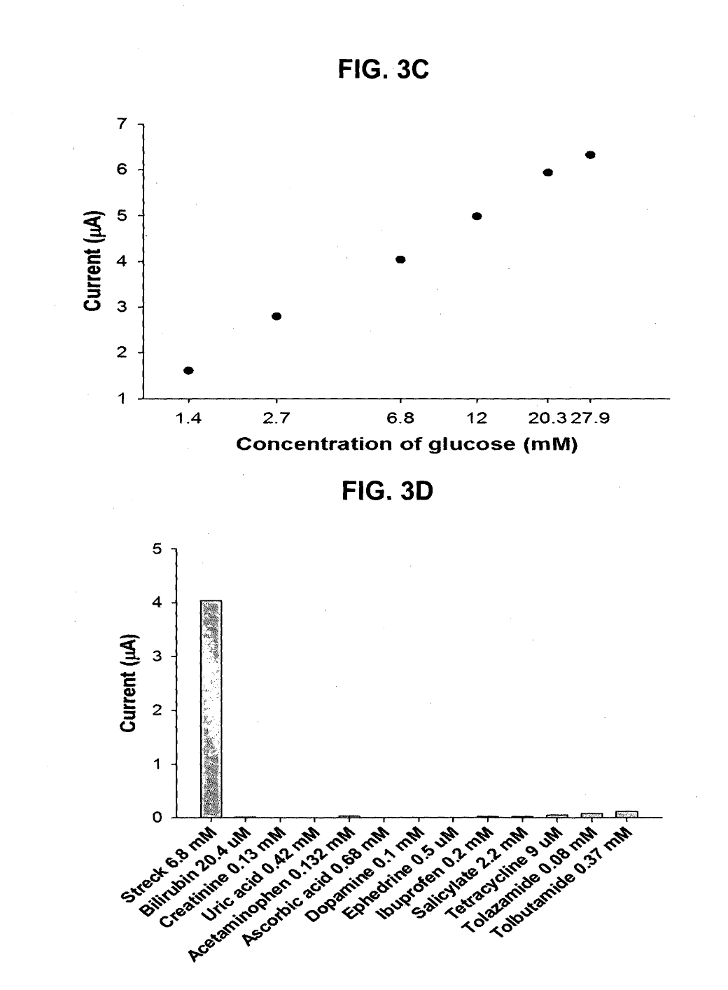

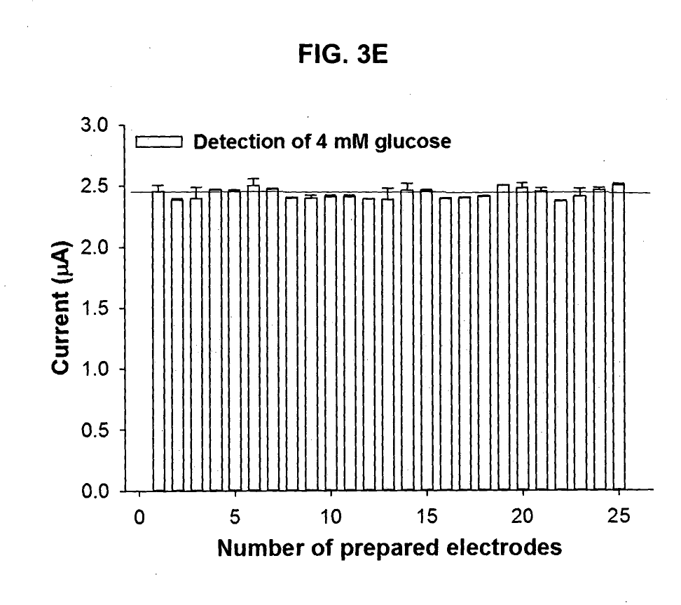

As shown in FIG. 3, Glucose assay curve was obtained on Nafion/GOx-APTES/GCE, Nafion/GOx/APTES/GCE land Nafion/GOx/APTES/GCE 2 by injecting varying volumes of 1 M glucose stock solution into the stirred PBS to form the final concentrations of 0.5, 1, 2, 4, 8, 16 and 32 mM in a 2 mL solution. All the concentrations were detected individually in triplicate.

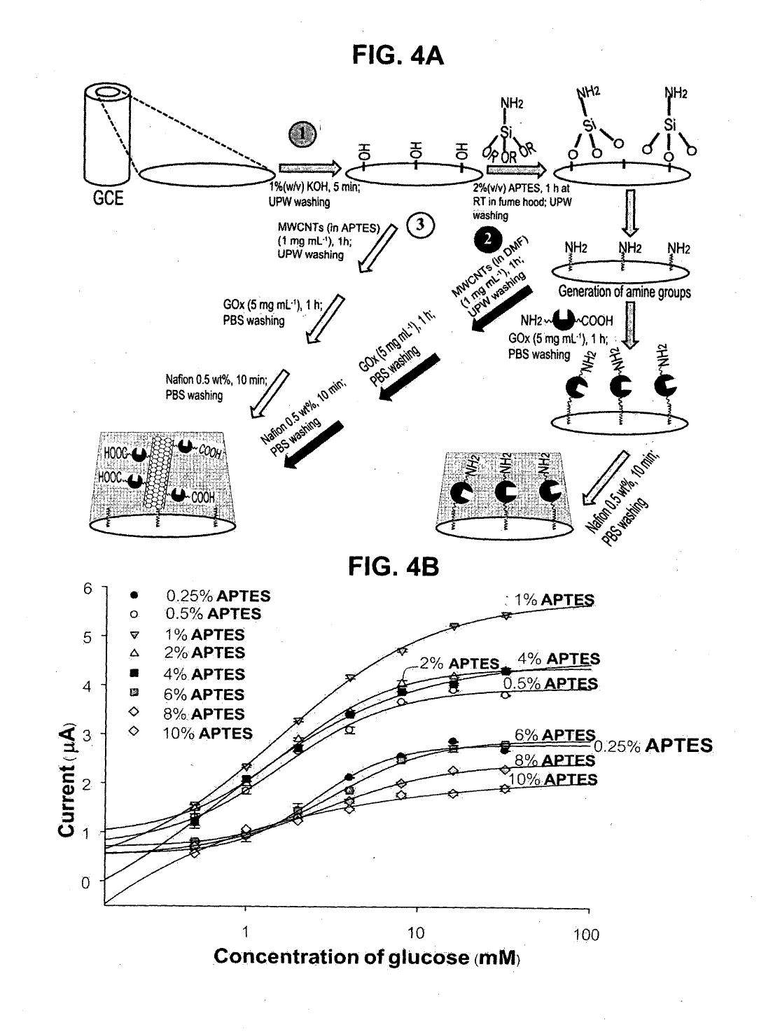

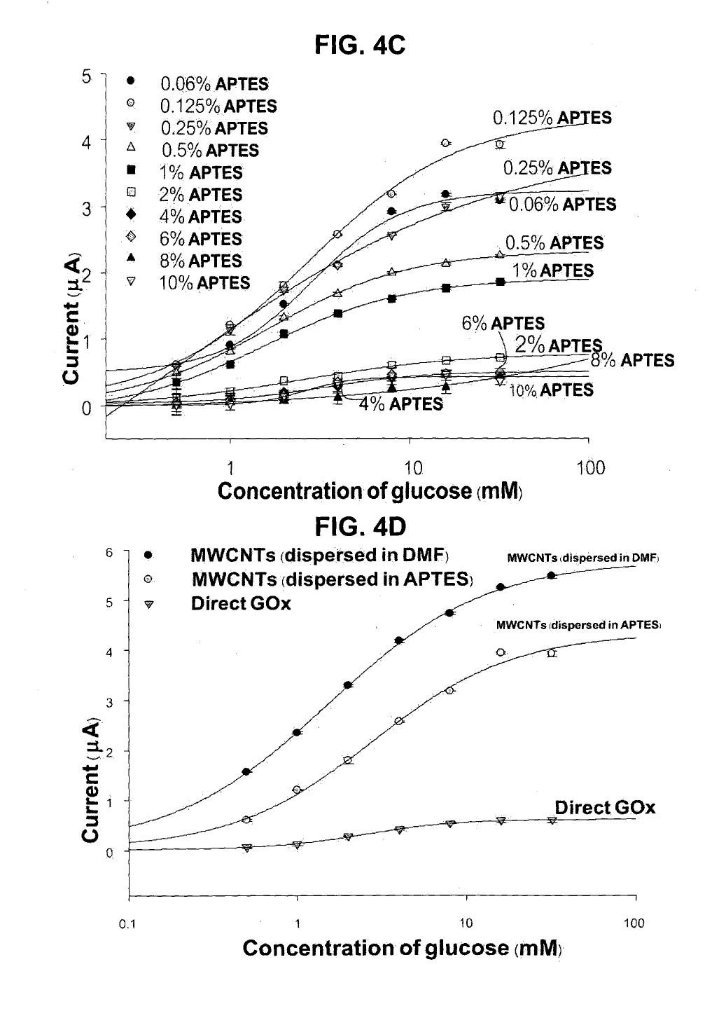

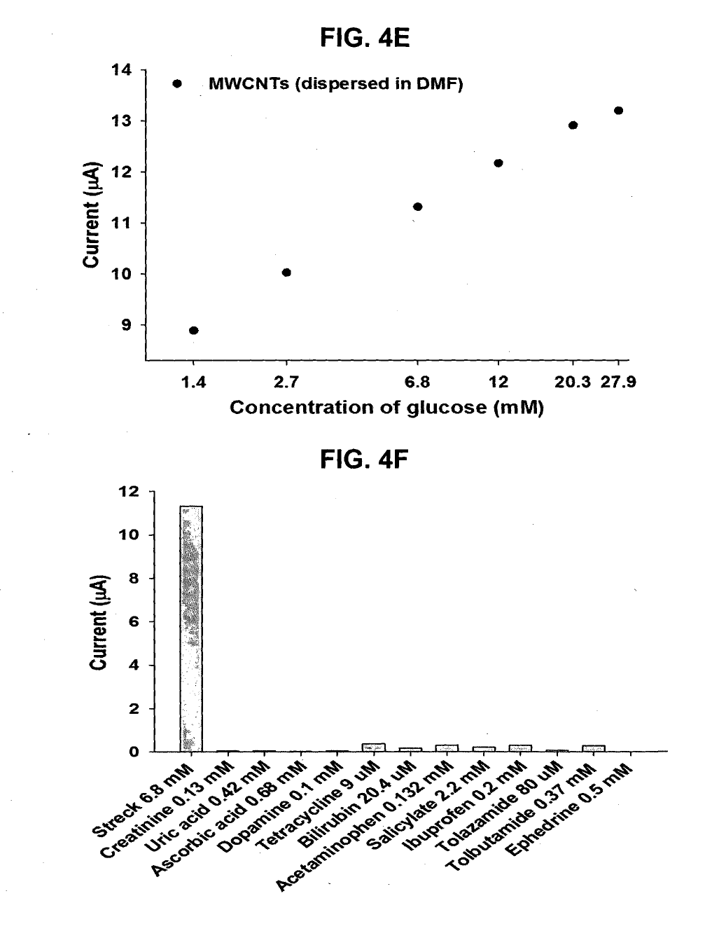

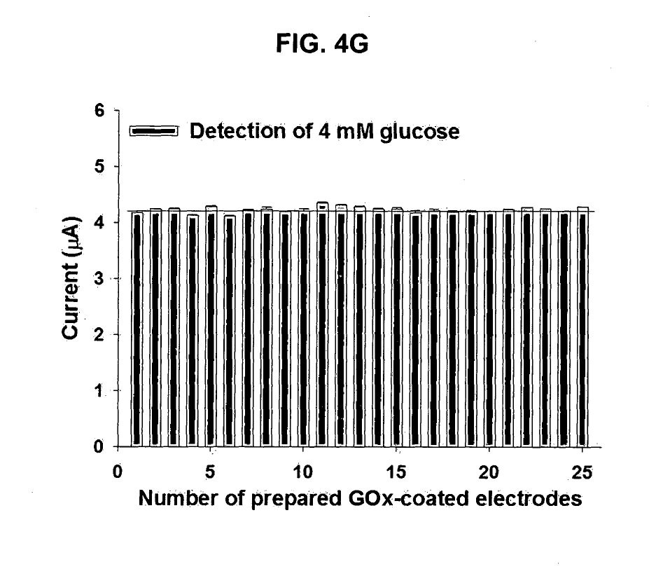

As shown in FIG. 4, streck assay curve was obtained on Nafion/GOx-APTES/GCE by injecting 400 μL of Sugar-Chex blood glucose linearity standards with different glucose concentrations, i.e. 1.3, 2.8, 6.6, 11.8, 20.3 and 28.2 mM, into 2.8 mL of stirred PBS.

(ii) Effect of Interfering Substances

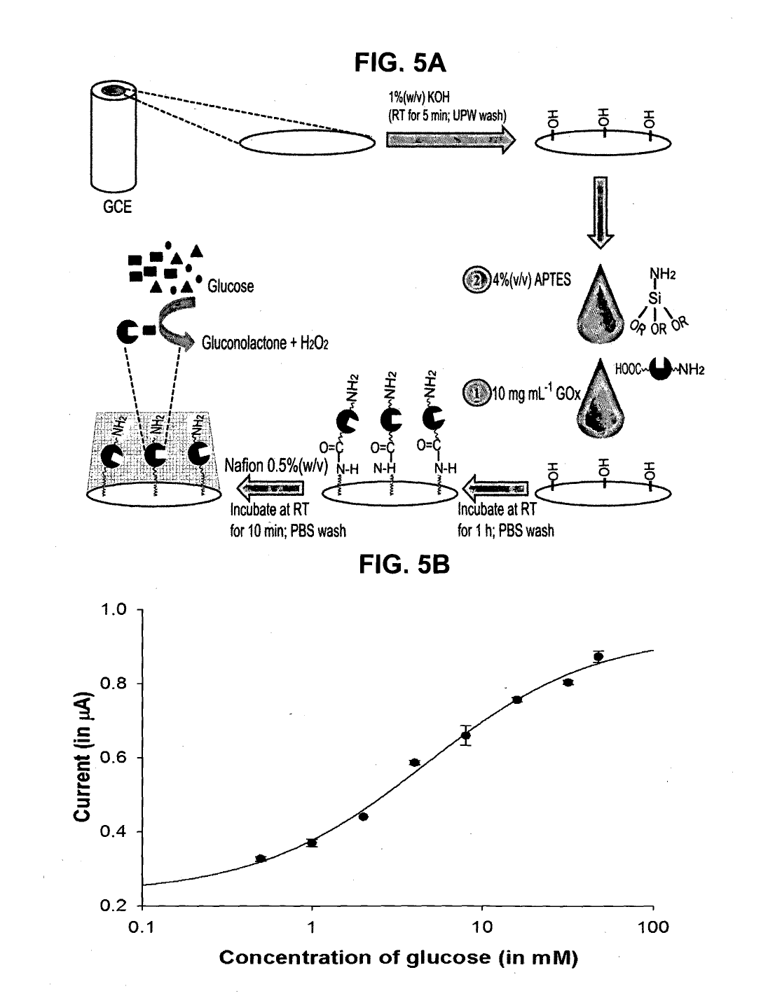

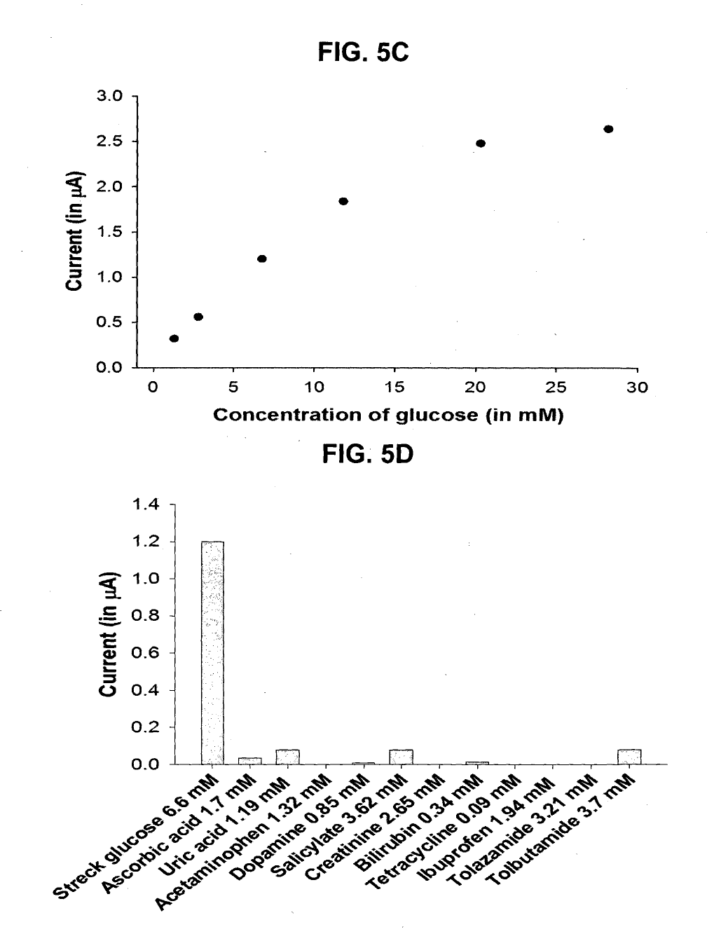

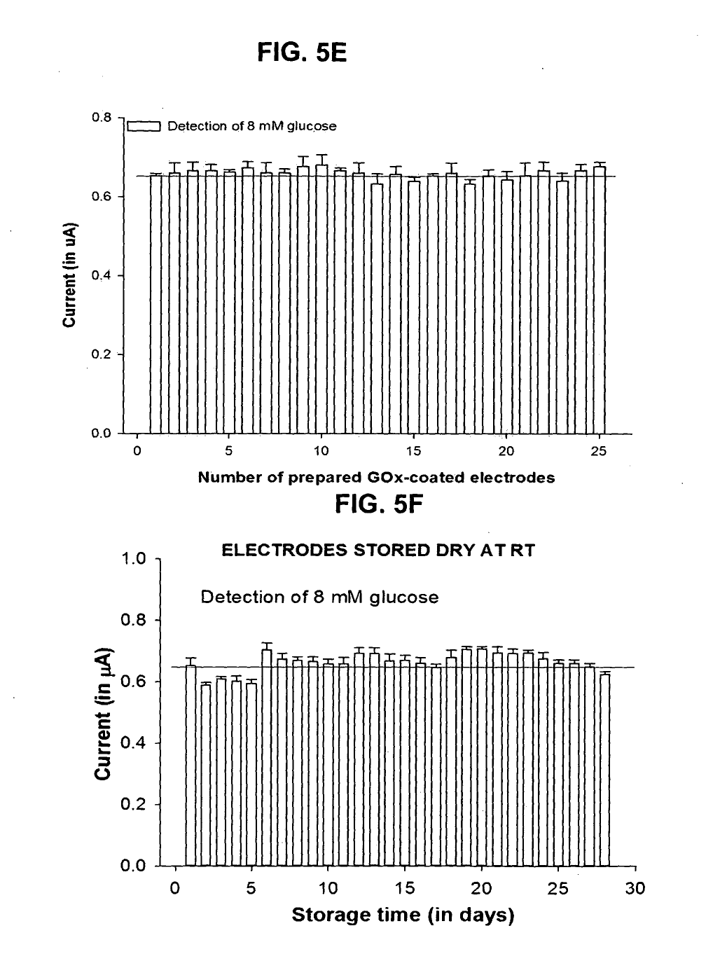

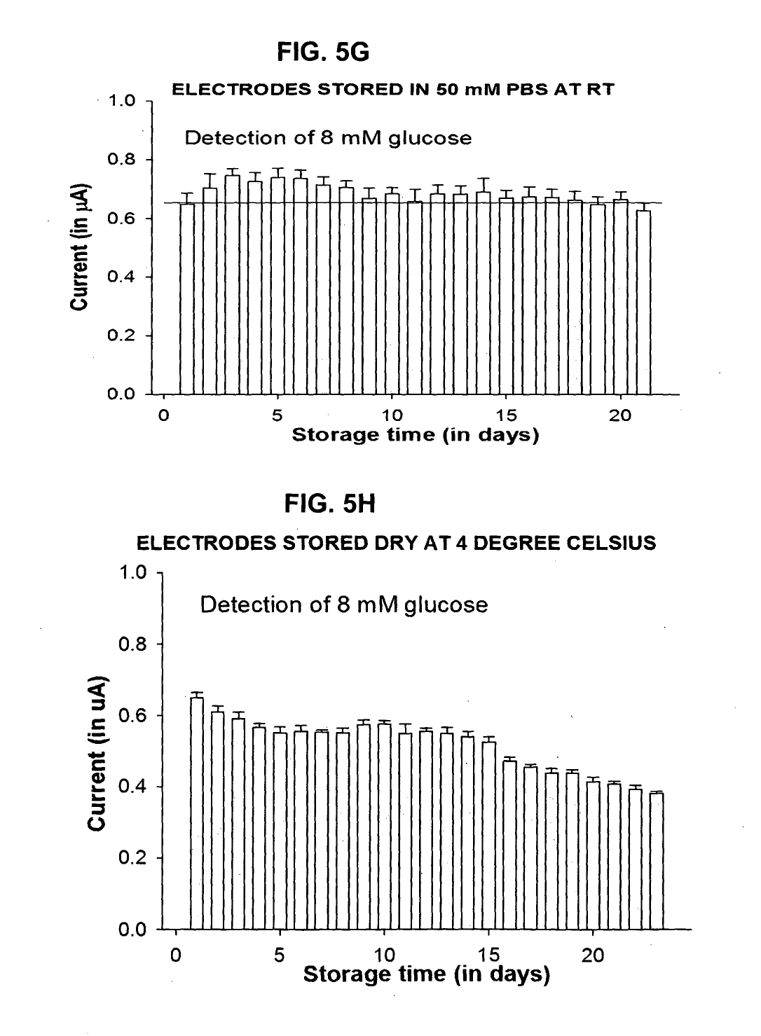

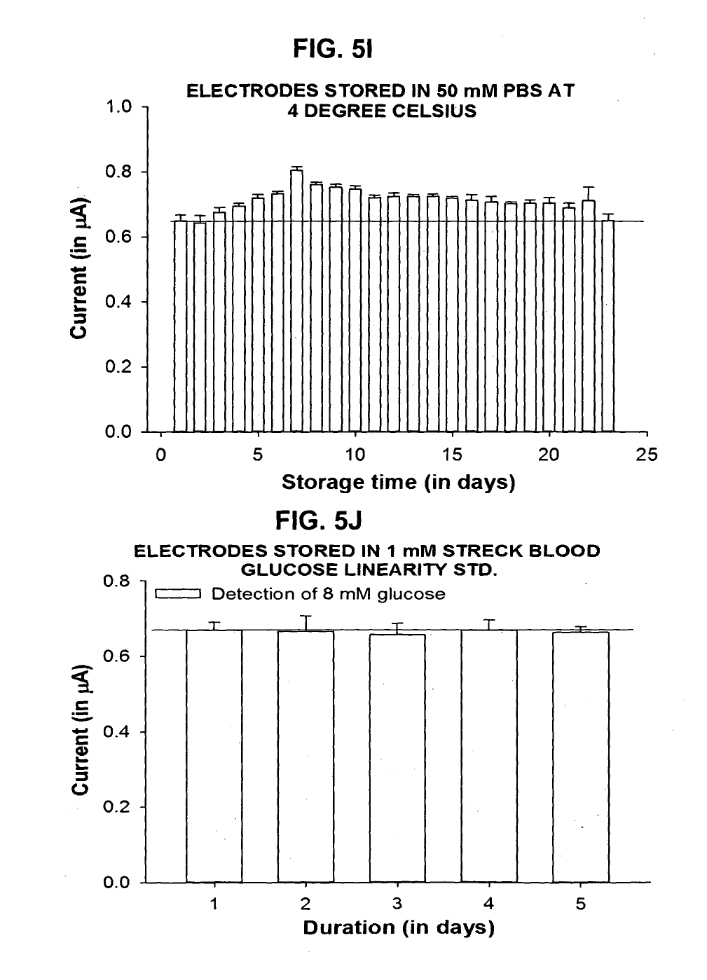

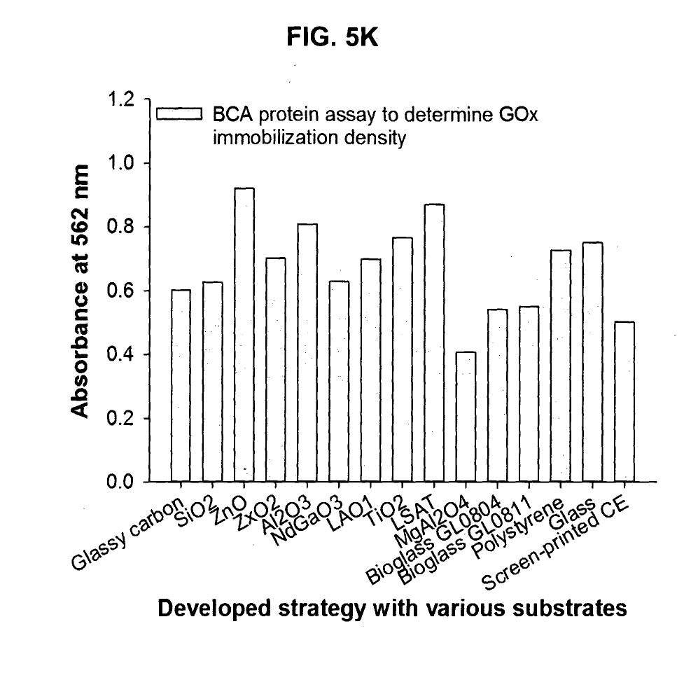

As shown in FIG. 5, ascorbic acid (0.28 M), dopamine (0.33 M), (+)-ephedrin-hydrochloride (4.96 mM) and creatinine (0.44 M) solution were prepared in 50 mM PBS. Uric acid solution (5.9 mM) and bilirubin (17 mM) were prepared in 10 mM NaOH solution. Tetracycline (2.25 mM) solution was prepared in 1 M HCl. Acetaminophen (0.33 M), salicylate (0.36 M), ibuprofen (48 mM) and tolbutamide (37 mM) solutions were prepared in absolute ethanol. Tolazamide solultion (32 mM) was prepared in acetone. The effect of interfering substances was determined by analyzing their effect on the electrochemical detection signal for 6.6 mM glucose after injection.

(iii) Continuous Glucose Monitoring

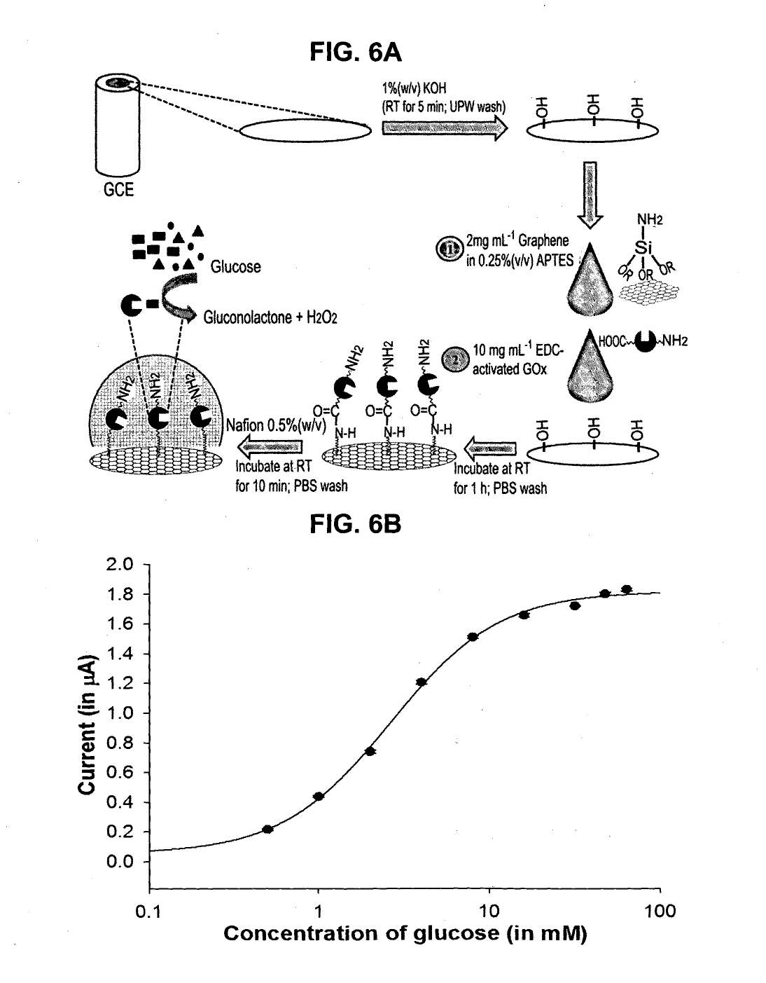

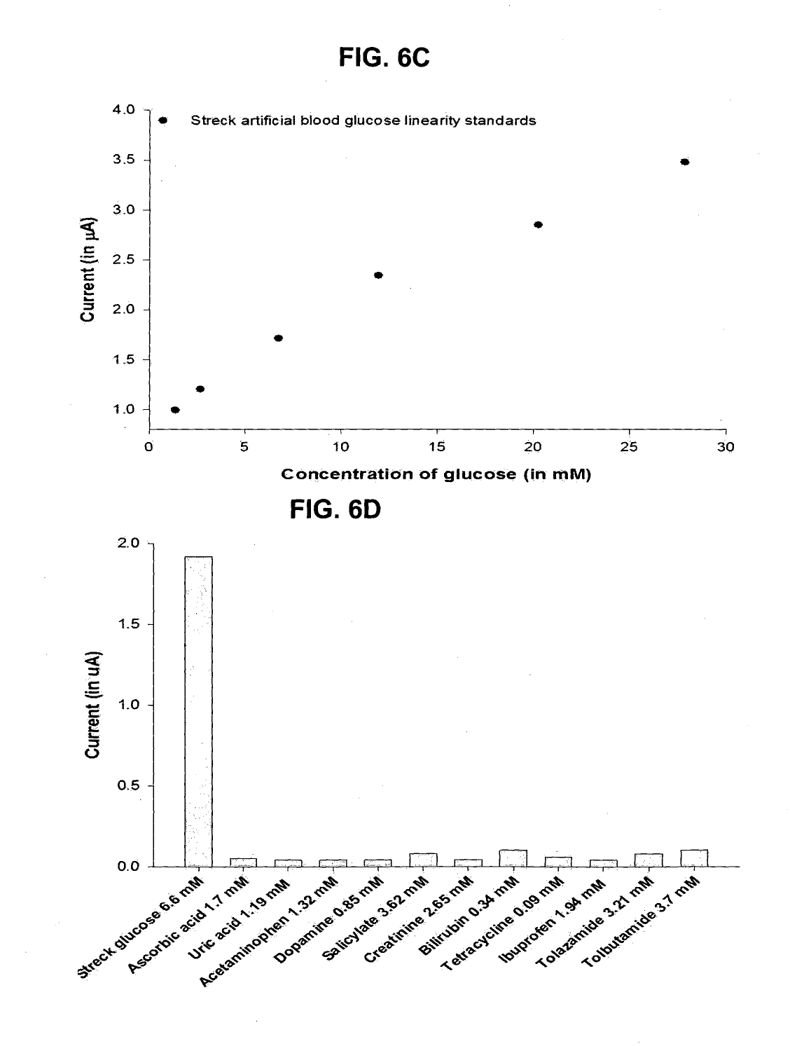

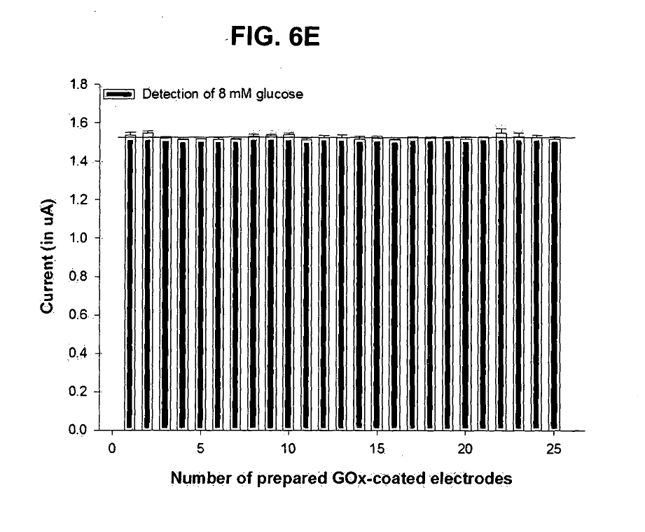

As shown in FIG. 6, the developed Nafion/GOx-APTES/GCE was used for continuous glucose monitoring, where 4 mM glucose was detected 150 times using the same electrode.

(iv) Effect of Biofouling

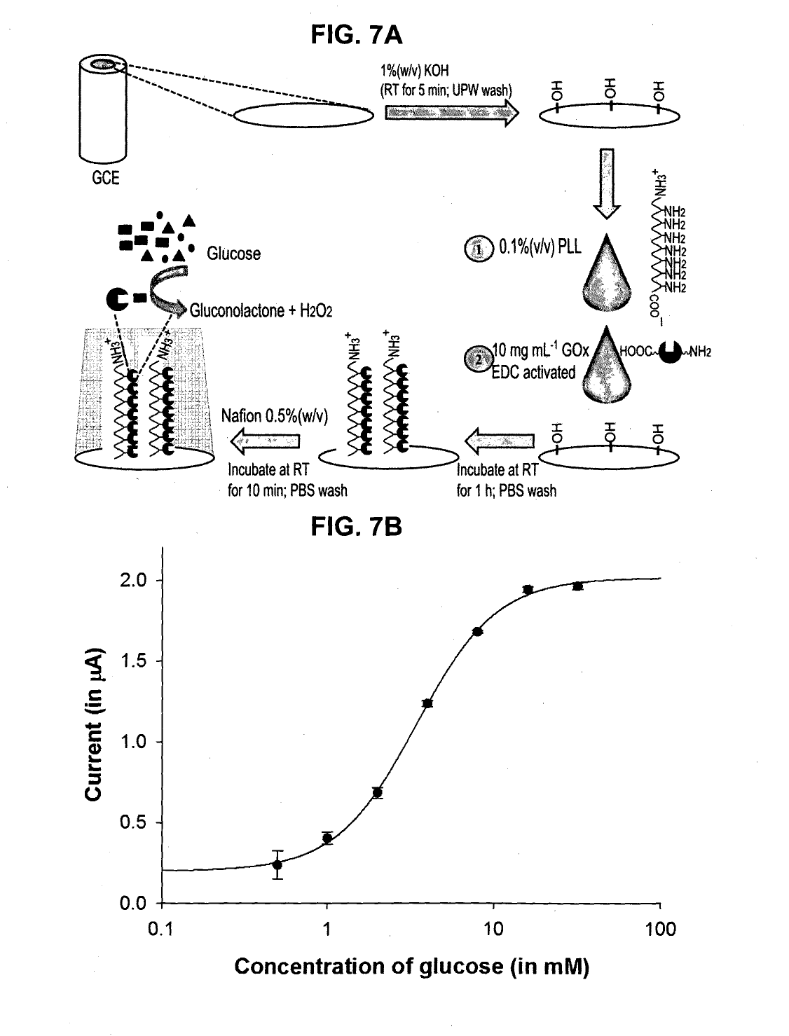

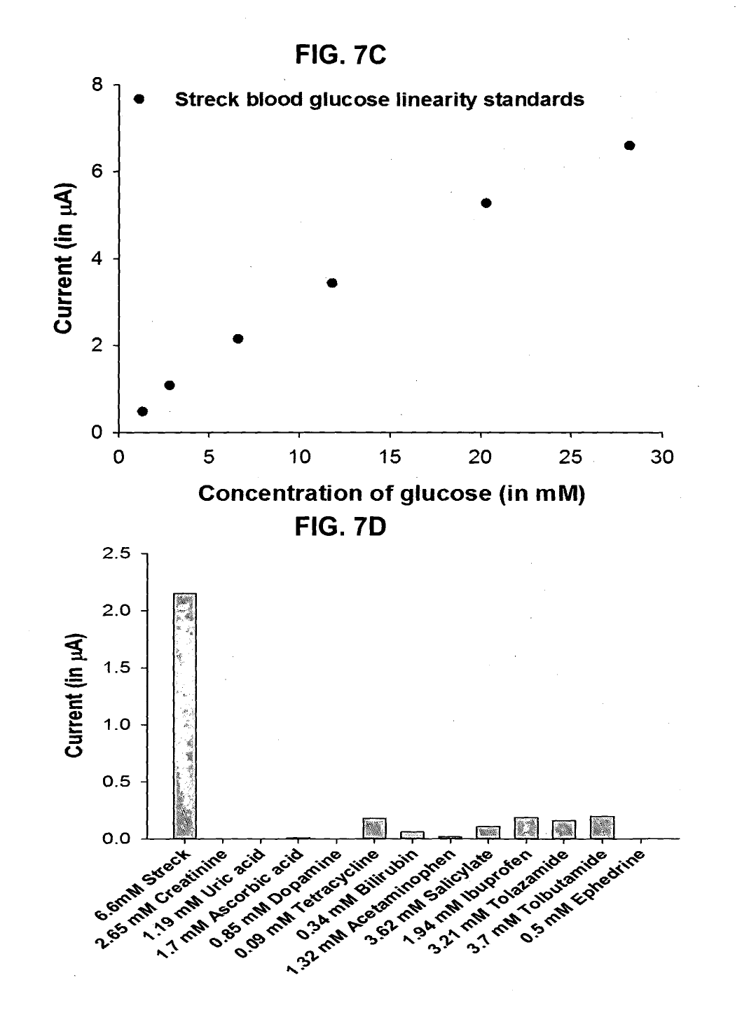

As shown in FIG. 7, the effect of biofouling was studied by the initial detection of 4 mM glucose on the freshly prepared Nafion/GOx-APTES/GCE, followed by the detection of 6.6 mM Streck blood glucose on the same electrode. This procedure was repeated four times.

(v) Production Reproducibility

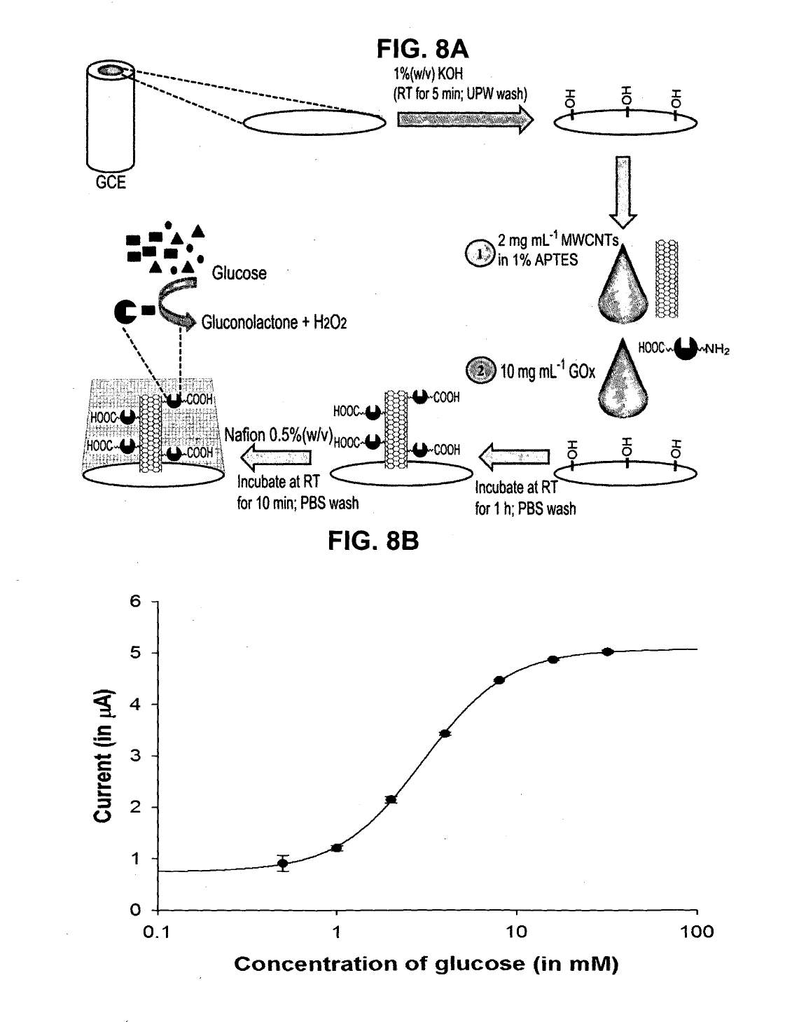

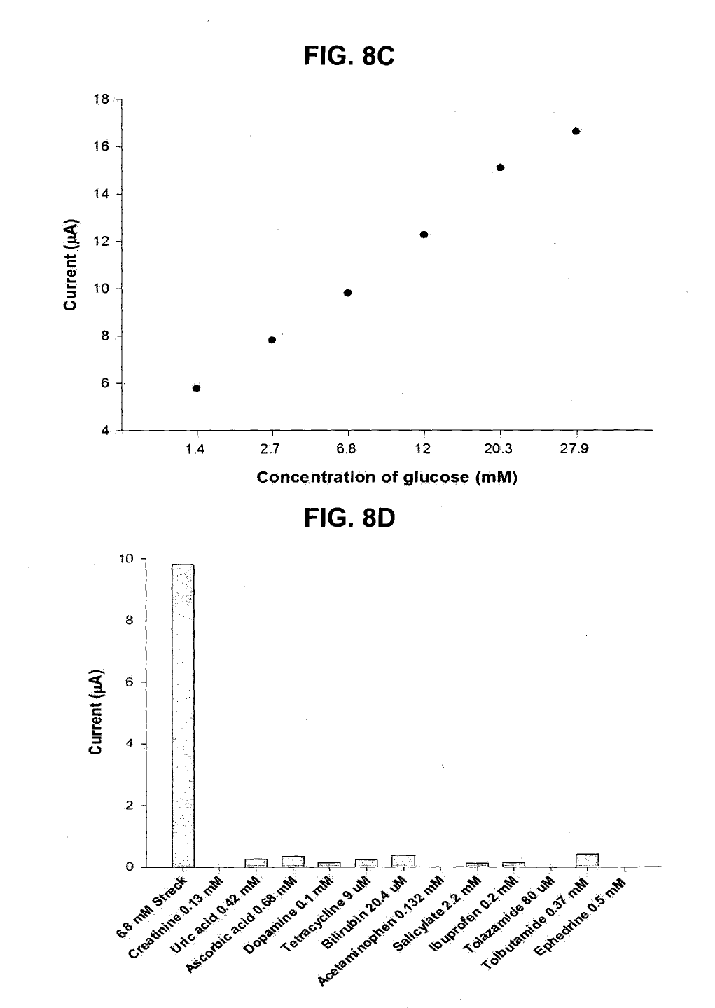

As shown in FIG. 8, the production reproducibility was determined from the reproducibility of electrochemical responses for the detection of 4 mM glucose (in triplicate) using 25 GOx-functionalized GCE prepared using the developed procedure.

1.2. Procedures and Methods for Employing Graphene Nano Platelets (GNPs)

1.2.1. Surface Cleaning and Generation of Hydroxyl Groups on GCE

Similar as mentioned in 1.1.1

1.2.2. Developed GNPs Based Multistep Strategy

As shown in FIG. 7, 1 mg of GNPs was mixed with 0.125% APTES and dispersed in ultrasonic bath for 1 h. 4 μL of the GNPs-APTES suspension was drop-casted on GCE surface and dried at RT for 1 h. Thereafter, the electrode was thoroughly washed with ultrapure water to form GNPs-APTES/GCE. 4 μL of EDC activated GOx (5 mg mL⁻¹) was drop casted on the GNPs-APTES/GCE and dried at RT for 1 h, after which the electrode was thoroughly washed with PBS to form GOx/GNPs-APTES/GCE. Finally, Nafion were coated using the similar procedure as mentioned in 1.2 to form Nafion/GOx/GNPs-APTES/GCE.

1.2.3. Electrochemical Analysis

Similar as mentioned in 1.1.4.

1.2.4. Detection of Glucose

(i) Assay Curve for Glucose (as Shown in FIG. 8)

Similar as mentioned in 1.1.5. (i).

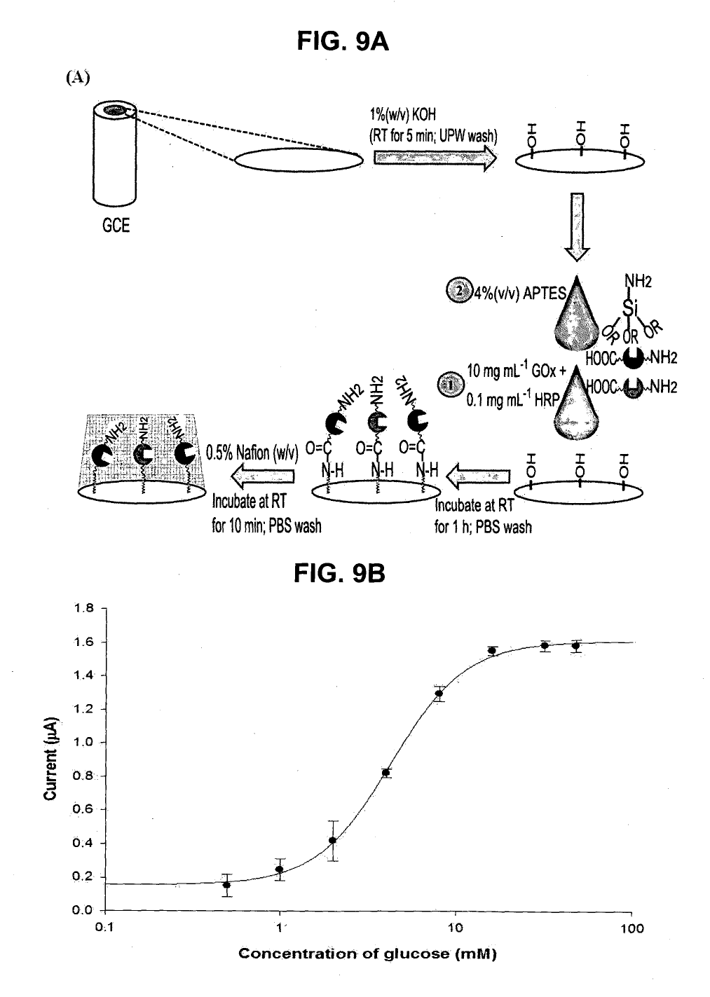

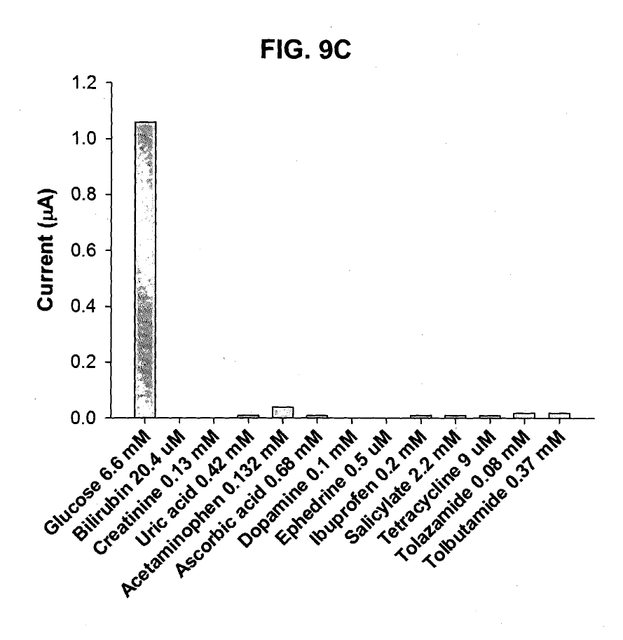

(ii) Effect of Interfering Substances (as Shown in FIG. 9)

Similar as mentioned in 1.1.5. (ii).

1.3. Procedures and Methods for Employing Poly-L-Lysine (PLL)

1.3.1. Surface Cleaning and Generation of Hydroxyl Groups on GCE

Similar as mentioned in 1.1.1

1.3.2. Developed EDC Crosslinked GOx Based Multistep Strategy

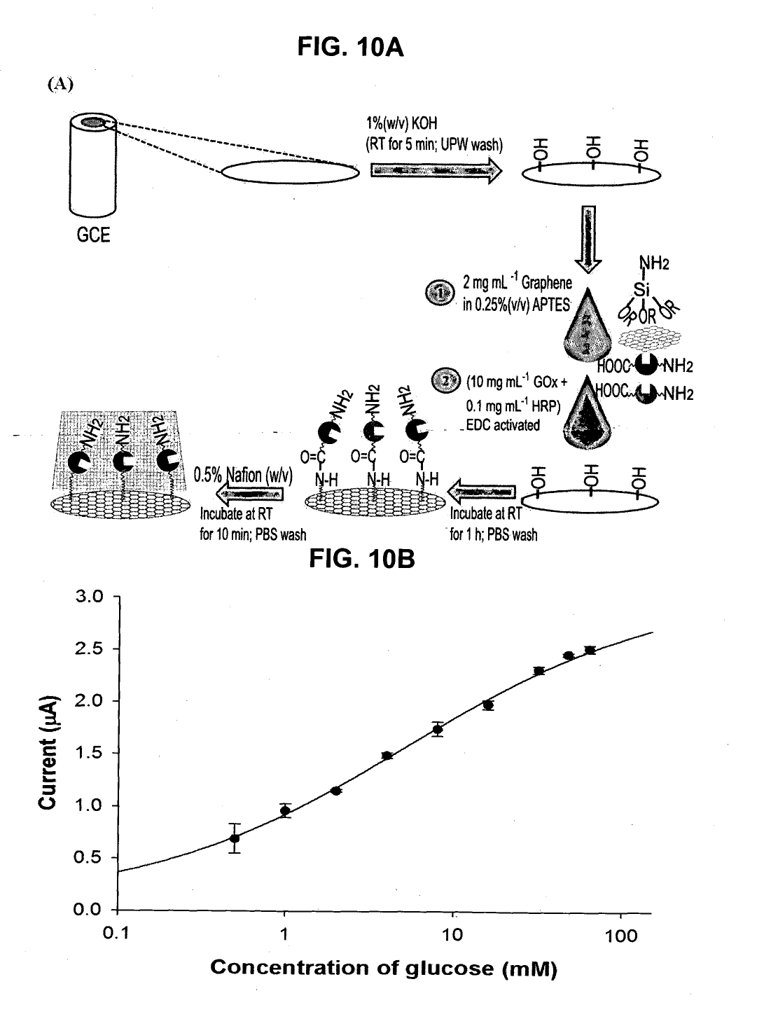

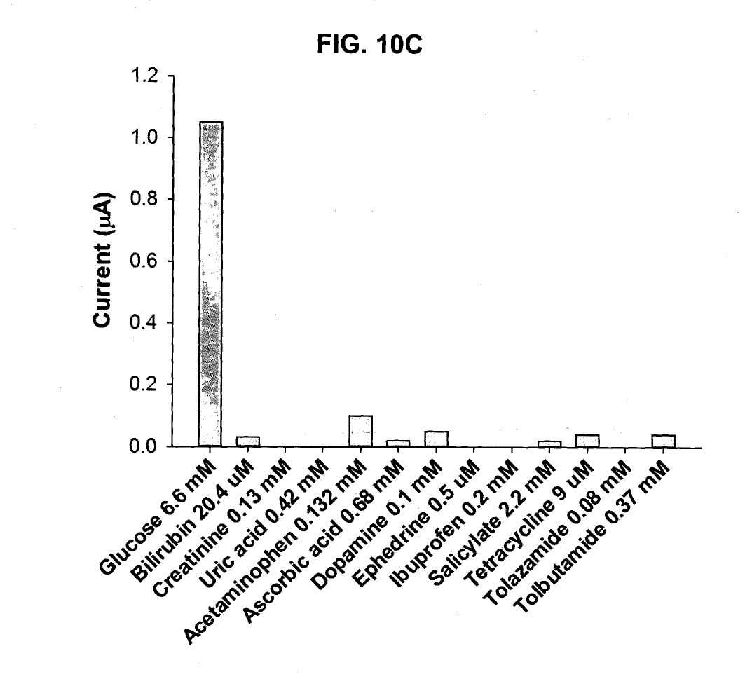

As shown in FIG. 10, 4 μL of the mixture of 0.1% PLL and 2% APTES was drop casted on GCE and dried at RT for 1 h followed by thoroughly washed by ultrapure water to form PLL-APTES/GCE. 4 μL of EDC crosslinked GOx (5 mg mL⁻¹) was drop casted on the PLL-APTES/GCE and dried at RT for 1 h followed by thoroughly washed with PBS to form GOx/PLL-APTES/GCE. Finally, Nafion were coated using the similar procedure as mentioned in 1.2 to form Nafion/GOx/PLL-APTES/GCE.

1.3.3. Electrochemical Analysis

Similar as mentioned in 1.1.4.

1.3.4. Detection of Glucose

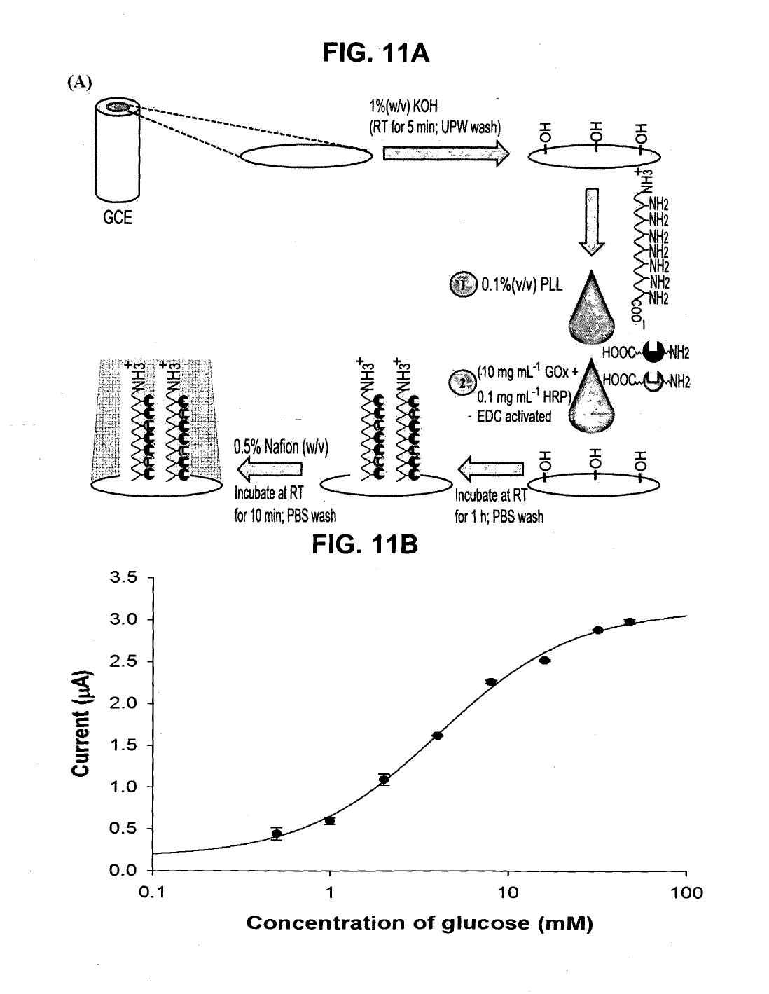

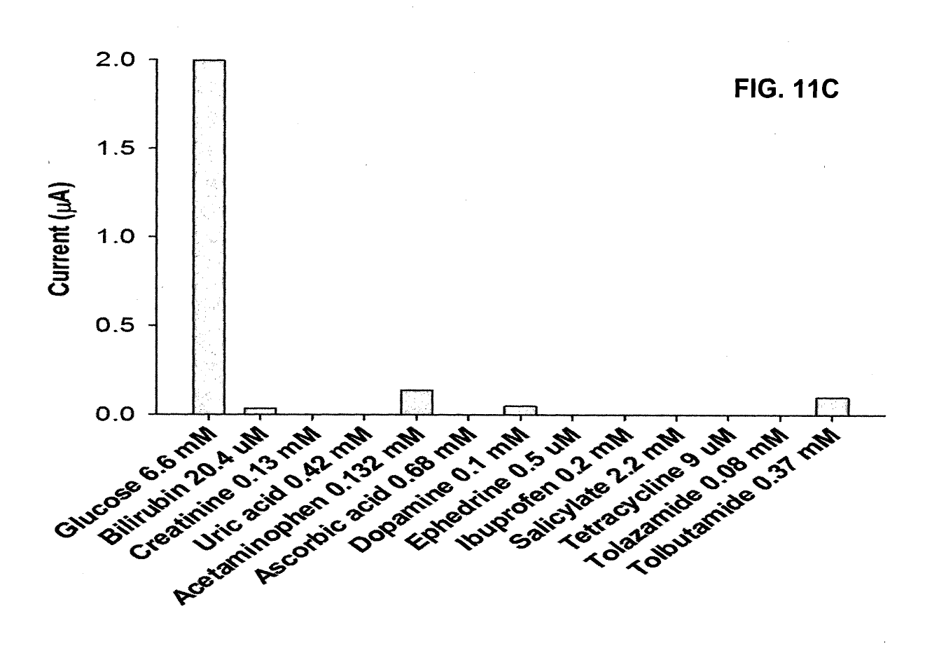

(i) Assay Curve for Glucose (as Shown in FIG. 11)

Similar as mentioned in 1.1.5. (i).

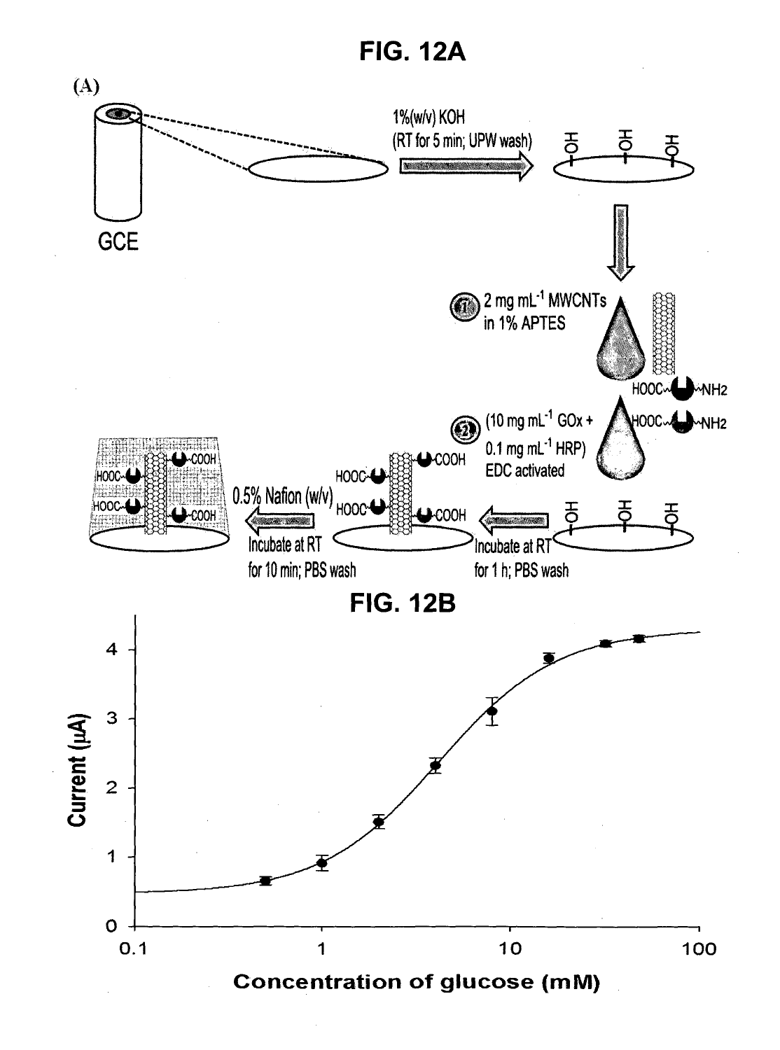

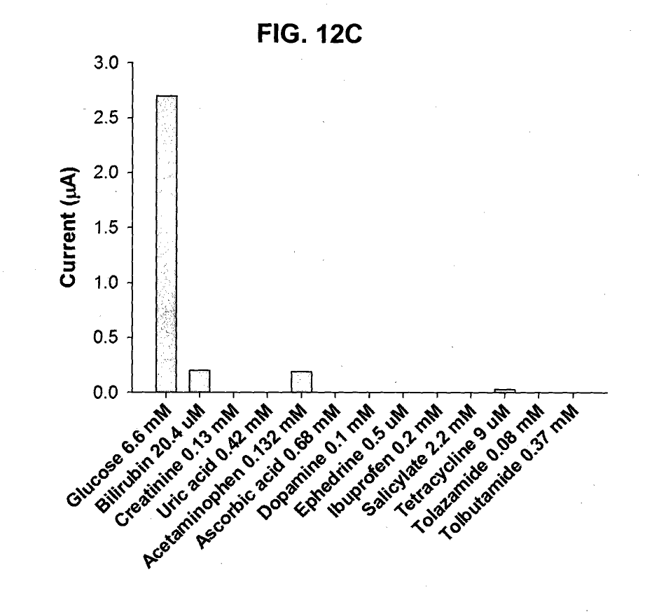

(ii) Effect of Interfering Substances (as Shown in FIG. 12)

Similar as mentioned in 1.1.5. (ii).

1.4. Procedures and Methods for Employing MWCNTs (Dispersed in APTES) Based Strategies)

1.4.1. Surface Cleaning and Generation of Hydroxyl Groups on GCE

Similar as mentioned in 1.1.1

1.4.2. Developed MWCNTs Based Strategy

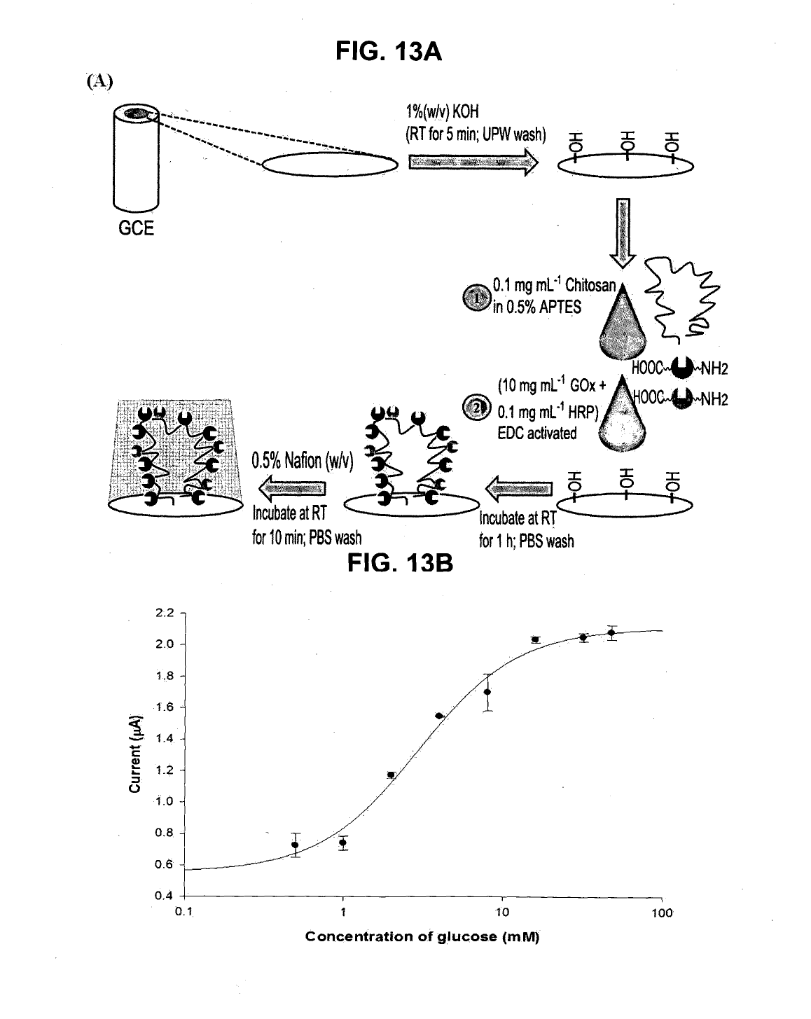

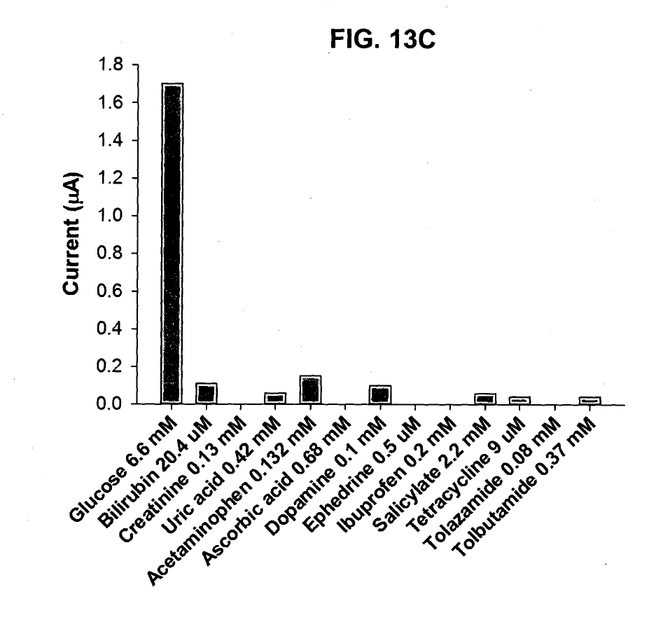

As shown in FIG. 13, 1 mg mL⁻¹ MWCNTs were dispersed in 0.25% APTES by keeping in an ultrasonic bath for 30 min. Then, 4 μL of the resulting MWCNTs-APTES solution was then drop cast on a GCE surface and dried at RT to form MWCNTs-APTES/GCE. Thereafter, 4 μL of 5 mg mL⁻¹ GOx was drop casted on the MWCNTs-APTES/GCE surface and dried at RT for 1 h followed by thoroughly washed with PBS to form GOx/MWCNTs-APTES/GCE. Finally, Nafion were coated using the similar procedure as mentioned in 1.2 to form Nafion/GOx/MWCNTs-APTES/GCE.

1.4.3. Electrochemical Analysis

Similar as mentioned in 1.1.4

1.4.4. Detection of Glucose

(i) Assay Curve for Glucose (as Shown in FIG. 14)

Similar as mentioned in 1.1.5. (i).

(ii) Effect of Interfering Substances (as Shown in FIG. 15)

Similar as mentioned in 1.1.5. (ii).

### Experiment 2

### A Highly-Simplified Procedure for the Preparation of Highly Stable And Lead-Proof Glucose-Oxidase Bound Electrodes

TABLE 4
Stability of the developed GOx-bound GCE, stored under
different conditions, in terms of the electrochemical
signal response for the detection of 8 mM glucose.

| Signal | | RT in 50 mM | | 4° C. in 50 mM |
|---|---|---|---|---|
| Strength | RT dry | PBS, pH 7.4 | 4° C. dry | PBS, pH 7.4 |
| No dec in | 27^{th} day | 20^{th} day | Dec. con- | 23^{rd} day |
| signal | | | tinuously | |
| 20% dec in | 40^{th} day | 38^{th} day | 15^{th} day | 33^{rd} day |
| signal | | | | |
| 25% dec in | 42^{nd} day | 41^{st} day | 16^{th} day | 41^{st} day |
| signal | | | | |
| 50% dec in | 62^{nd} day | 61^{st} day | 38^{th} day | 60^{th} day |
| signal | | | | |

TABLE 5
Comparison of the developed highly-simplified glucose sensing strategies with
various leading commercial glucose meters.

| | | | | LifeScan | | Graphene | | |
|---|---|---|---|---|---|---|---|---|
| | Abbott | Bayer | Roche Accu- | one | | Nano | Poly-L- | |
| Analytical | Freestyle | Contour | Chek | Touch | | Platelets- | lysine- | MWCNTs- |
| Parameters | Lite | USB | Advantage | Ultra 2 | GOx | GOx | GOx | GOx |
| Dynamic | 2-28 | 2-28 | 2-28 | 2-28 | 0.5-48 | 0.5-80 | 0.5-32 | 0.5-16 |
| range | Problems | Problems | Problems | Problems | | | | |
| | >22 & <2 | >24 & <2 | >22 & <2 | >22 & <2 | | | | |
| Assay time | 5 s | 5 s | 5 s | 5 s | 4 s | 4 s | 4 s | 4 s |
| Mediator | Os | Ferricyanide | Ferricyanide | Ferricyanide | None | None | None | None |
| added | complex | | | | | | | |
| Enzymes | GDH | GDH | GDH | GOx | GOx | GOx | GOx | GOx |
| Precision | <5% | <5% | <5% | <5% | <5% | <5% | <5% | <5% |
| Interferences | No | N.M. | 30 μg/mL | 30 μg/mL | No | No | No | No |
| | | | Ascorbic | Ascorbic | | | | |
| | | | acid, 20 μg/mL | acid | | | | |
| | | | Acetaminophen, | | | | | |
| | | | 40 μg/mL | | | | | |
| | | | Dopamine | | | | | |
| Drugs | N.M. | N.M. | N.M. | N.M. | No | No | No | No |
| Potential | −0.16 V | 0.4 V | 0.4 V | 0.4 V | −0.45 V | −0.45 V | −0.45 V | −0.45 V |
| Multisubstrate- | No | No | No | No | Yes | Yes | Yes | Yes |
| compatible | | | | | | | | |

TABLE 6
Comparison of the developed highly-simplified glucose sensing strategies with
various leading commercial continuous glucose monitoring systems.

| | Noninvasive: | | | | | | | |
|---|---|---|---|---|---|---|---|---|
| | GlucoWatch | Subcutaneous: | Subcutaneous: | | | | | |
| | G2 | Freestyle | Guardian | | | Graphene | Poly- | |
| | Biographer | Navigator | REAL-Time | Subcutaneous: | | Nano | L- | |
| Analytical | (Johnson & | (Abbott | (Medtronic | DexCom | | Platelets- | lysine- | MWCNTs- |
| Parameters | Johnson) | Diabetes) | MiniMed) | STS CGMS | GOx | GOx | GOx | GOx |
| Dynamic | 2-22 | 2-22 | 2-22 | 2-22 | 0.5-48 | 0.5-80 | 0.5-32 | 0.5-16 |
| range | Problems | Problems | Problems | Problems | | | | |
| | >22 & <2 | >24 & <2 | >22 & <2 | >22 & <2 | | | | |
| Assay time | N.M. | N.M. | N.M. | N.M. | 4 s | 4 s | 4 s | 4 s |
| Mediator | None | Os | None | None | None | None | None | None |
| added | | complex | | | | | | |
| Enzymes | GOx | GOx | GOx | GOx | GOx | GOx | GOx | GOx |
| Precision | <5% | <5% | <5% | <5% | <5% | <5% | <5% | <5% |
| Interferences | N.M. | No | N.M. | N.M. | No | No | No | No |
| Drugs | N.M. | N.M. | N.M. | N.M. | No | No | No | No |
| Potential | 0.42 V | −0.2 V | N.M. | N.M. | −0.45 V | −0.45 V | −0.45 V | −0.45 V |
| Sensor life | 1/2 | 5 | 3 | 7 | >7 | In | In | In |
| span (days) | | | | | | progress | progress | progress |
| Multisubstrate- | No | No | No | No | Yes | Yes | Yes | Yes |
| compatible | | | | | | | | |

Materials and Equipment Used

Material and equipment used in this experiment are shown in Table 7.

TABLE 7

| Description | Company | Cat. No. |
|---|---|---|
| 3-Aminopropyltriethoxysilane | Sigma | A3648 |
| Glucose oxidase | Sigma | G7141 |
| D-glucose | Sigma | G7528 |
| 70 wt. % Glutaraldehyde | Sigma | G7776 |
| 5 wt. % Nafion | Sigma | 527084 |
| Ascorbic acid | Sigma | A5960 |
| Uric acid | Sigma | U2625 |
| Acetaminophen | Sigma | A7085 |
| Dopamine hydrochloride | Sigma | H8502 |
| Creatinine | Sigma | C4255 |
| Bilirubin | Sigma | B4126 |
| Salicylic acid | Sigma | 247588 |
| Tetracycline | Sigma | 268054 |
| Bilirubin | Sigma | B4126 |
| Ibuprofen | Sigma | I4883 |
| Tolazamide | Sigma | T2408 |
| Tolbutamide | Sigma | T0891 |
| (+)-Ephedrin-hydrochloride | Sigma | 857335 |
| Hydrochloric acid | Sigma | 320331 |
| Sodium hydroxide | Sigma | S8045 |
| Acetone | Sigma | 179124 |
| Ethanol | Sigma | 459844 |
| Poly-L-lysine | Sigma | P8920 |
| BupH phosphate buffered saline | Thermo Scientific | 28372 |
| BupH MES buffered saline | Thermo Scientific | 28390 |
| 1-Ethy-(3-dimethylaminopropyl) | Thermo Scientific | 22981 |
| carbodiimide•HCl | | |
| BCA Protein Assay kit | Thermo Scientific | 23227 |
| Sugar-Chex Linearity, Low | Streck, Inc. | 290311 |
| 12345 | (USA) | |
| Graphene Nano Platelets | CheapTubes. | Grade 2 |
| | Inc. (USA) | |
| Multi-walled carbon nanotubes | NanoLab, Inc. | PD15LL1-5 |
| | (USA) | |
| Glassy carbon working electrode | CH Instruments, | CHI104 |
| | Inc (USA) | |
| Platinum wire counter electrode | CH Instruments, | CHI115 |
| | Inc | |
| Silver/silver chloride reference | CH Instruments, | CHI111 |
| electrode | Inc | |
| CHI660A electrochemical | CH Instruments, | CHI660A |
| workstation | Inc | |
| BASi C3 Cell Stand | BASi | EF-1085 |
| EDP3-plus electronic pipettes | Rainin | E3-20 |
| with LTS, 2-20 μL | | |
| EDP3-plus electronic pipettes | Rainin | E3-200 |
| with LTS, 20-200 μL | | |
| EDP3-plus electronic pipettes | Rainin | E3-1000 |
| with LTS, 100-1000 μL | | |
| Ultrasonic cleaner (Model: | Branson | B2510E-MT |
| 2510) | | CPN-952-236 |
| Eppendorf microtubes | Sigma | Z 606340 |
| Nunc microwell 96-well poly- | Sigma | P7491 |
| styrene plates | | |
| ELISA Plate Reader | Tecan | 30050303 |
| Thermomixer comfort | Eppendorf | EPPE5355000.038 |
| Water purification system | Millipore | ZRQSVP0UK |
| (Model: Direct Q) | | |
| SigmaPlot software | Systat | version 11.2 |

50 mM PBS was used as a diluent for GOx and glucose dilutions, and also for washings after the process steps (as specified below) in the developed procedure. GOx stock solution, prepared by mixing equal volumes of 20 mg mL⁻¹ GOx and 5% glutaraldehyde, was stored at 4° C. and used for experiments after equilibrating for 30 mM at RT.

2.1 Procedures or Methods for Directing GOx Binding

2.1.1. Surface Cleaning and Generation of Hydroxyl Groups on GCE

Same as mentioned in 1.1.1

2.1.2. Developed Simplified Strategy

2 μL of 10 mg mL⁻¹ GOx was drop-casted on. GCE followed by immediate drop-casting of 2 μL of 4% (w/v) APTES to form APTES-GOx mixture on GCE. The APTES-GOx/GCE was dried at room temperature (RT) for 1 h, washed extensively with 50 mM PBS and then drop-casted with 3 μL of 0.5% Nafion to form Nafion/APTES-GOx/GCE followed by extensive washing with 50 mM PBS. The developed strategy was also employed on platinum (Pt) and gold (Au) electrodes to fabricate Nafion/APTES-GOx/PtE and Nafion/APTES-GOx/AuE.

A variation of the developed strategy was also employed for comparison, where 4% APTES was first drop-casted on GCE followed by the addition of 10 mg mL⁻¹ GOx solution. The electrode modified by this varied strategy is denoted as Nafion/GOx-APTES/GCE.

2.1.3. Electrochemical Analysis

All electrochemical measurements were done at RT on CHI 660A electrochemical workstation using three electrode system i.e. developed working electrode, Pt counter electrode and Ag/AgCl reference electrode. The amperometric response of glucose was recorded in stirred PBS at −450 mV vs. 3 M Ag/AgCl.

2.1.4. Detection of Glucose

(i) Assay Curve for Glucose and Streck's Sugar-Chex Blood Glucose Linearity Standards

Glucose assay curve was obtained on Nafion/APTES-GOx/GCE by injecting varying volumes of 1 M glucose stock solution into the stirred PBS to form the final concentrations of 0.5, 1, 2, 4, 8, 16, 32 and 48 mM in a 2 mL solution. All the concentrations were detected individually in triplicate. Streck assay curve was obtained by injecting 400 microliters of Sugar-Chex blood glucose linearity standards, with different glucose concentrations, i.e. 1.3, 2.8, 6.6, 11.8, 20.3 and 28.2 mM, into 2.8 mL of stirred PBS.

(ii) Effect of Interfering Substances

Ascorbic acid (0.28 M), dopamine (0.33 M) and creatinine (0.44 M) solutions were prepared in 50 mM PBS. Uric acid solution (5.9 mM) was prepared in 10 mM NaOH. Tetracycline (2.25 mM) and bilirubin (17 mM) solutions were prepared in 1 M HCl. Acetaminophen (0.33 M), salicylate (0.36 M), ibuprofen (48 mM) and tolbutamide (37 mM) solutions were prepared in absolute ethanol. Tolazamide solultion (32 mM) was prepared in acetone. The effect of interfering substances was determined by analyzing the effect of injecting consecutively the stated concentrations of various interfering substances on the electrochemical detection signal for 6.6 mM of Sugar-Chex glucose linearity standard.

(iii) Reproducibility for Preparing GOx-Bound GCE Using the Developed Simplified Procedure

The developed simplified procedure was used for preparing 25 GOx-bound GCEs. The production reproducibility was then determined by the electrochemical detection of 8 mM glucose (in triplicate) on each electrode.

(iv) Stability of Developed GOx-Bound Electrodes Stored Under Various Conditions

The stability of the developed GOx-bound electrodes was assessed under four storage conditions that are being widely used in biomedical diagnostics.
Storage in 50 mM PBS at 4° C.: The developed Nafion/APTES-GOx/GCE was employed for detecting 8 mM glucose ten times each day from the time it was freshly prepared (corresponding to 100% signal strength) to about 2 months (when the signal strength decreased to 50%). The electrode was stored in 50 mM PBS at 4° C.
Storage in Dry state (without PBS) at 4° C.: The developed Nafion/APTES-GOx/GCE was employed for detecting 8 mM glucose ten times each day from the time it was freshly prepared (corresponding to 100% signal strength) to about 5 weeks (when the signal strength decreased to 50%). The electrode was stored in dry state (without PBS) at 4° C.
Storage in 50 mM PBS at RT: The developed Nafion/APTES-GOx/GCE was employed for detecting 8 mM glucose ten times each day from the time it was freshly prepared (corresponding to 100% signal strength) to about 2 months (when the signal strength decreased to 50%). The electrode was stored in 50 mM PBS at 4° C.
Storage in Dry state (without PBS) at RT: The developed Nafion/APTES-GOx/GCE was employed for detecting 8 mM glucose tem times each day from the time it was freshly prepared (corresponding to 100% signal strength) to about 2 months (when the signal strength decreased to 50%). The electrode was stored in dry state (without PBS) at 4° C.

(v) Effect of Storing the Developed GOx-Bound Electrode in Streck's Blood Glucose Linearity Standard for 5 Days

The Nafion/APTES-GOx/GCE was stored overnight at RT dipped in Streck's Sugar-Chex blood glucose linearity standard (1 mM). The biofouling was determined by taking the electrochemical signals of Nafion/APTES-GOx/GCE for detecting 8 mM glucose immediately after preparing GOx-bound electrode and every day after storing in Streck's blood glucose for 5 days.

2.1.5 Demonstration of the Multisubstrate-Compatibility of the Developed Simplified Strategy for Binding GOx on Different Substrates

The developed simplified strategy was employed for binding GOx on different types of substrates of exactly same area. Bicinchoninic acid (BCA) protein assay was then performed to determine the concentration of GOx bound to the various substrates. APTES-GOx coated substrates were incubated in 200 microliters of BCA reagent for 30 min at 37° C. (using the Thermomixer comfort). Thereafter, 180 microliters of purple-colored BCA protein assay solution, resulting from the reaction of bound GOx on various substrates with the BCA reagent, was transferred to a 96-well microtiter plate whose absorbance was taken at 562 nm.

2.2. Procedure and Method for Employing Graphene Nano Platelets

2.2.1. Surface Cleaning and Generation of Hydroxyl Groups on GCE

Same as mentioned in 1.1.1.

2.2.2. Developed Highly-Simplified Strategy

2 μL of 2 mg mL⁻¹ graphene nano platelets (GNPs; diameter 5 μm) dispersed in 0.25% APTES were drop-casted on GCE followed by immediate drop-casting of 2 μL of 10 mg mL⁻¹ GOx to form APTES-GNPs-GOx mixture on GCE. The APTES-GNPs-GOx/GCE was dried at RT for 1 h and washed extensively with 50 mM PBS. Thereafter, it was drop-casted with 3 μL of 0.5% Nafion and dried at RT for 10 min to form Nafion/APTES-GNPs-GOx/GCE followed by extensive washing with 50 mM PBS.

2.2.3. Electrochemical Analysis

Same as mentioned in 2.1.3.

2.2.4. Assay Curve for Glucose

Glucose assay curve was obtained on Nafion/APTES-GNPs-GOx/GCE by injecting varying volumes of 1 M glucose stock solution into the stirred PBS to form final concentrations of 0.5, 1, 2, 4, 8, 16, 32, 48 and 64 mM in a 2 mL solution. All the concentrations were detected individually in triplicate.

Streck assay curve was obtained using the same procedure as mentioned in 2.1.4. (i).

2.2.5. Effect of Interfering Substances

Same as mentioned in 2.1.4. (ii).

2.2.6. Reproducibility for Preparing Nafion/APTES-GNPs-GOx/GCE Using the Developed Simplified Procedure

The developed simplified procedure was used for preparing 25 Nafion/APTES-GNPs-GOx/GCE. The production reproducibility was then determined by the electrochemical detection of 8 mM glucose (in triplicate) on each electrode.

2.3. Procedure and Method for Employing Poly-L-Lysine

2.3.1. Surface Cleaning and Generation of Hydroxyl Groups on GCE

Same as mentioned in 2.1.1.

2.3.2. Developed Highly-Simplified Strategy Employing EDC Based Cross-Linking

0.12 g mL⁻¹ of 1-Ethy-(3-dimethylaminopropyl) carbodiimide (EDC) was prepared in 100 mM MES. 2 μL of 0.1% poly-L-lysine (PLL) was drop-casted initially on cleaned GCE followed by immediate drop-casting of 2 μL of 10 mg mL⁻¹ GOx (activated by EDC for 15 min before use) to form PLL-GOx mixture on GCE. The PLL-GOx/GCE was dried at RT for 1 h and washed extensively with 50 mM PBS. Thereafter, it was drop-casted with 3 μL of 0.5% Nafion and dried at RT for 10 min to form Nafion/PLL-GOx/GCE followed by extensive washing with 50 mM PBS.

2.3.3. Electrochemical Analysis

Same as mentioned in 2.1.3.

2.3.4. Detection of Glucose

(i) Assay Curve for Glucose and Streck's Sugar-Chex Blood Glucose Linearity Standards

Glucose assay curve was obtained on Nafion/PLL-GOx/GCE by injecting varying volumes of 1 M glucose stock solution into the stirred PBS to form final concentrations of 0.5, 1, 2, 4, 8, 16 and 32 mM in a 2 mL solution. All the concentrations were detected individually in triplicate.

Streck assay curve was obtained using the same procedure as mentioned in 2.1.4. (i).

(ii) Effect of Interfering Substances

Same as mentioned in 2.1.4. (ii).

2.4. Procedure and Method for Employing Multi-Walled Carbon Nanotubes

2.4.1. Surface Cleaning and Generation of Hydroxyl Groups on GCE

Same as mentioned in 2.1.1.

2.4.2. Developed highly-simplified strategy

2 μL of 2 mg mL⁻¹ multi-walled carbon nanotubes (MWCNTs) (diameter 15 nm and length 1-5 μm) dispersed in 1% APTES were drop-casted on GCE followed by the immediate drop-casting of 2 μL of 10 mg mL⁻¹ GOx to form APTES-MWCNTs-GOx mixture on GCE. The APTES-MWCNTs-GOx/GCE was dried at RT for 1 h and then washed extensively with 50 mM PBS. Thereafter, it was drop-casted with 3 μL of 0.5% Nafion and dried at RT for 10 min to form Nafion/APTES-MWCNTs-GOx/GCE followed by extensive washing with 50 mM PBS

2.4.3. Electrochemical Analysis

Same as mentioned in 2.1.3.

2.4.4. Assay Curve for Glucose

Same as mentioned in 2.3.4. (i).

2.4.5. Effect of Interfering Substances

Same as mentioned in 2.1.4. (ii).

### Experiment 3

### A Bienzyme-Based Mediator-Less Electrochemical Glucose Sensing Strategy

TABLE 8
Comparison of the developed bienzyme-based EC glucose sensing strategies
with the leading commercial glucose meters.

| | Abbott | Bayer | Roche Accu- | LifeScan | | Graphene | Poly- | | |
|---|---|---|---|---|---|---|---|---|---|
| Analytical | Freestyle | Contour | Chek | one Touch | | Nano | L- | | |
| Parameters | Lite | USB | Advantage | Ultra 2 | GOx | Platelets | lysine | MWCNTs | Chitosan |
| Dynamic | 2-28 | 2-28 | 2-28 | 2-28 | 0.5-16 | 0.5-64 | 0.5-48 | 0.5-48 | 0.5-16 |
| range | Problems | Problems | Problems | Problems | | | | | |
| | >22 & <2 | >24 & <2 | >22 & <2 | >22 & <2 | | | | | |
| Assay | 5 s | 5 s | 5 s | 5 s | 4 s | 4 s | 4 s | 4 s | 4 s |
| time | | | | | | | | | |
| Mediator | Os complex | Ferricyanide | Ferricyanide | Ferricyanide | None | None | None | None | None |
| added | | | | | | | | | |
| Enzymes | GDH | GDH | GDH | GOx | GOx- | GOx-HRP | GOx- | GOx- | GOx- |
| | | | | | HRP | | HRP | HRP | HRP |
| Precision | <5% | <5% | <5% | <5% | <5% | <5% | <5% | <5% | <5% |
| Interferences | No | N.M. | 30 μg/mL | 30 μg/mL | No | No | No | No | No |
| | | | Ascorbic | Ascorbic | | | | | |
| | | | acid, 20 μg/mL | acid | | | | | |
| | | | Acetaminophen, | | | | | | |
| | | | 40 μg/mL | | | | | | |
| | | | Dopamine | | | | | | |
| Drugs | N.M. | N.M. | N.M. | N.M. | No | No | No | No | No |
| Potential | −0.16 V | 0.4 V | 0.4 V | 0.4 V | −0.45 V | −0.45 V | −0.45 V | −0.45 V | −0.45 V |
| Multisubstrate- | No | No | No | No | Yes | Yes | Yes | Yes | Yes |
| compatible | | | | | | | | | |

TABLE 9
Comparison of the developed bienzyme-based EC glucose sensing strategies
with the leading commercial continuous glucose monitoring systems.

| | Noninvasive: | | | | | | | | |
|---|---|---|---|---|---|---|---|---|---|
| | GlucoWatch | Subcutaneous: | Subcutaneous: | | | | | | |
| | G2 | Freestyle | Guardian | | | | | | |
| | Biographer | Navigator | REAL-Time | Subcutaneous: | | Graphene | Poly- | | |
| Analytical | (Johnson & | (Abbott | (Medtronic | DexCom | | Nano | L- | | |
| Parameters | Johnson) | Diabetes) | MiniMed) | STS CGMS | GOx | Platelets | lysine | MWCNTs | Chitosan |
| Dynamic | 2-22 | 2-22 | 2-22 | 2-22 | 0.5-48 | 0.5-64 | 0.5-48 | 0.5-48 | 0.5-16 |
| range | Problems | Problems | Problems | Problems | | | | | |
| | >22 & <2 | >24 & <2 | >22 & <2 | >22 & <2 | | | | | |
| Assay | N.M. | N.M. | N.M. | N.M. | 4 s | 4 s | 4 s | 4 s | 4 s |
| time | | | | | | | | | |
| Mediator | None | Os complex | None | None | None | None | None | None | None |
| added | | | | | | | | | |
| Enzymes | GOx | GOx | GOx | GOx | GOx- | GOx-HRP | GOx- | GOx- | GOx- |
| | | | | | HRP | | HRP | HRP | HRP |
| Precision | <5% | <5% | <5% | <5% | <5% | <5% | <5% | <5% | <5% |
| Interferences | N.M. | No | N.M. | N.M. | No | No | No | No | No |
| Drugs | N.M. | N.M. | N.M. | N.M. | No | No | No | No | No |
| Potential | 0.42 V | −0.2 V | N.M. | N.M. | −0.45 V | −0.45 V | −0.45 V | −0.45 V | −0.45 V |
| Multisubstrate- | No | No | No | No | Yes | Yes | Yes | Yes | Yes |
| compatible | | | | | | | | | |

Materials and Equipment Used

Material and equipment used in this experiment are shown in Table 10.

TABLE 10

| Description | Company | Cat. No. |
|---|---|---|
| 3-Aminopropyltriethoxysilane | Sigma | A3648 |
| Glucose oxidase | Sigma | G7141 |
| Peroxidase from horseradish | Sigma | P8375 |
| D-glucose | Sigma | G7528 |
| 70 wt. % Glutaraldehyde | Sigma | G7776 |
| 5 wt. % Nafion | Sigma | 527084 |
| Ascorbic acid | Sigma | A5960 |
| Uric acid | Sigma | U2625 |
| Acetaminophen | Sigma | A7085 |
| Dopamine hydrochloride | Sigma | H8502 |
| Creatinine | Sigma | C4255 |
| Bilirubin | Sigma | B4126 |
| Salicylic acid | Sigma | 247588 |
| Tetracycline | Sigma | 268054 |
| Bilirubin | Sigma | B4126 |
| Ibuprofen | Sigma | I4883 |
| Tolazamide | Sigma | T2408 |
| Tolbutamide | Sigma | T0891 |
| (+)-Ephedrin-hydrochloride | Sigma | 857335 |
| Hydrochloric acid | Sigma | 320331 |
| Sodium hydroxide | Sigma | S8045 |
| Acetone | Sigma | 179124 |
| Ethanol | Sigma | 459844 |
| Poly-L-lysine | Sigma | P8920 |
| Chitosan | Sigma | C3646 |
| BupH phosphate buffered saline | Thermo Scientific | 28372 |
| BupH MES buffered saline | Thermo Scientific | 28390 |
| 1-Ethy-(3-dimethylaminopropyl) | Thermo Scientific | 22981 |
| carbodiimide•HCl | | |
| Sugar-Chex Linearity, Low | Streck, Inc. | 290311 |
| 12345 | (USA) | |
| Graphene Nano Platelets | CheapTubes. | Grade 2 |
| | Inc. (USA) | |
| Multi-walled carbon nanotubes | NanoLab, Inc. | PD15LL1-5 |
| | (USA) | |
| Glassy carbon working electrode | CH Instruments, | CHI104 |
| | Inc (USA) | |
| Platinum wire counter electrode | CH Instruments, | CHI115 |
| | Inc | |
| Silver/silver chloride reference | CH Instruments, | CHI111 |
| electrode | Inc | |
| CHI660A electrochemical | CH Instruments, | CHI660A |
| workstation | Inc | |
| BASi C3 Cell Stand | BASi | EF-1085 |
| EDP3-plus electronic pipettes | Rainin | E3-20 |
| with LTS, 2-20 μL | | |
| EDP3-plus electronic pipettes | Rainin | E3-200 |
| with LTS, 20-200 μL | | |
| EDP3-plus electronic pipettes | Rainin | E3-1000 |
| with LTS, 100-1000 μL | | |
| Ultrasonic cleaner (Model: | Branson | B2510E-MT |
| 2510) | | CPN-952-236 |
| Eppendorf microtubes | Sigma | Z 606340 |
| Nunc microwell 96-well poly- | Sigma | P7491 |
| styrene plates | | |
| ELISA Plate Reader | Tecan | 30050303 |
| Thermomixer comfort | Eppendorf | EPPE5355000.038 |
| Water purification system | Millipore | ZRQSVP0UK |
| (Model: Direct Q) | | |
| SigmaPlot software | Systat | version 11.2 |

50 mM PBS was used as a diluent for GOx and glucose dilutions, and also for washings after the process steps (as specified below) in the developed procedure. 30 microliters of bienzyme solution 1, prepared by mixing equal volumes of 20 mg mL⁻¹ GOx and 0.2 mg mL⁻¹ HRP, was mixed with 2 microliters of 0.12 g mL⁻¹ 1-ethy-(3-dimethylaminopropyl) carbodiimide (EDC, dissolved in MES) for 15 min at room temperature (RT) before use. Bienzyme solution 2 was prepared by mixing equal volumes of 20 mg mL⁻¹ GOx (in 2.5% glutaraldehyde) and 0.2 mg mL⁻¹ HRP for 15 min at RT before use.

3.1 Procedures or Methods for Directing GOx binding

3.1.1. Surface Cleaning and Generation of Hydroxyl Groups on GCE

Same as mentioned in 1.1.1

3.1.2. Developed Highly-Simplified Strategy

2 μL of bienzyme solution 2 was drop-casted on GCE followed by immediate drop-casting of 2 μL of 4% APTES to form APTES-GOx-HRP mixture on GCE. The APTES-GOx-HRP/GCE was dried at RT for 1 h and then washed extensively with 50 mM PBS. Thereafter, it was drop-casted with 3 μL of 0.5% Nafion and dried at RT for 10 min to form Nafion/APTES-GOx-HRP/GCE followed by extensive washing with 50 mM PBS.

3.1.3. Electrochemical Analysis

All electrochemical measurements were done at RT on CHI 660A electrochemical workstation using a three electrode system, i.e., developed working electrode, Pt counter electrode and 3 M Ag/AgCl reference electrode. The amperometric response of glucose was recorded in stirred PBS at −450 mV vs. Ag/AgCl.

(i) Assay Curve for Glucose

Glucose assay curve was obtained on Nafion/PLL-GOx-HRP/GCE by injecting varying volumes of 1 M glucose stock solution into the stirred PBS to form the final concentrations of 0.5, 1, 2, 4, 8, 16, 32 and 48 mM in a 2 mL solution. All the concentrations were detected individually in triplicate.

(ii) Effect of Interfering Substances

Bilirubin (5.1 mM) and uric acid (11.9 mM) solutions were prepared in 10 mM NaOH. Creatinine (88.3 mM), acetaminophen (66 mM), ascorbic acid (0.57 M), dopamine (62.6 mM) and ephedrine (0.5 mM) solutions were prepared in 0.1 M PBS. Ibuprofen (48.6 mM), salicylate (0.36 M) and tolbutamide (37 mM) solutions were prepared in absolute ethanol. Tetracycline solution (4.5 mM) was prepared in 3 M HCl. Tolazamide solultion (32 mM) was prepared in acetone. The effect of interfering substances was determined by analyzing their effect on the electrochemical detection signal for 6.6 mM glucose after injection.

3.2 Procedures or Methods for Employing Graphene Nano Platelets

3.2.1. Surface Cleaning and Generation of Hydroxyl Groups on GCE

Same as mentioned in 1.1.1.

3.2.2 Developed highly-simplified GNPs based bienzyme strategy

2 μL of 2 mg mL⁻¹ graphene nano platelets (GNPs; diameter 5 μm) dispersed in 0.25% APTES were drop-casted on GCE followed by immediate drop-casting of 2 μL of bienzyme solution 1 to form GNPs-GOx-HRP mixture on GCE. The GNPs-GOx-HRP/GCE was dried at RT for 1 h and washed extensively with 50 mM PBS. Thereafter, it was drop-casted with 3 μL of 0.5% Nafion and dried at RT for 10 min to form Nafion/GNPs-GOx-HRP/GCE followed by extensive washing with 50 mM PBS.

3.2.3. Electrochemical Analysis

Same as mentioned in 3.1.3

3.2.4. Detection of Glucose

(i) Assay Curve for Glucose

Same as mentioned in 3.1.3. (i).

(ii) Effect of Interfering Substances

Same as mentioned in 3.1.3. (ii).

3.3 Procedures or Methods for Employing Poly-L-Lysine

3.3.1. Surface Cleaning and Generation of Hydroxyl Groups on GCE

Same as mentioned in 1.1.1.

3.3.2. Developed Simplified PLL Based Bienzyme Strategy

2 μL of 0.1% PLL was drop-casted on GCE followed by immediate drop-casting of 2 μL of bienzyme solution 1 to form PLL-GOx-HRP mixture on GCE. The PLL-GOx-HRP/GCE was dried at RT for 1 h, washed extensively with 50 mM PBS and then drop-casted with 3 μL of 0.5% Nafion to form Nafion/PLL-GOx-HRP/GCE followed by extensive washing with 50 mM PBS.

3.3.3. Electrochemical Analysis

Same as mentioned in 3.1.3.

3.3.4. Detection of Glucose

(i) Assay Curve for Glucose

Same as mentioned in 3.1.3. (i).

(ii) Effect of Interfering Substances

Same as mentioned in 3.1.3. (ii).

3.4 Procedures or Methods for Employing MWCNTs

3.4.1 Surface Cleaning and Generation of Hydroxyl Groups on GCE

Same as mentioned in 3.1.1.

3.4.2. Developed Highly-Simplified MWCNTs Based Bienzyme Strategy

2 μL of 2 mg mL⁻¹ MWCNTs (dispersed in 1% APTES) was drop-casted initially on cleaned GCE followed by immediate drop-casting of 2 μL of bienzyme solution 1 to form MWCNTs-GOx-HRP mixture on GCE. The MWCNTs-GOx-HRP/GCE was dried at RT for 1 h and washed extensively with 50 mM PBS. Thereafter, it was drop-casted with 3 μL of 0.5% Nafion and dried at RT for 10 mM to form Nafion/MWCNTs-GOx-HRP/GCE followed by extensive washing with 50 mM PBS.

3.4.3. Electrochemical Analysis

Same as mentioned in 3.1.3.

3.4.4. Detection of Glucose

(i) Assay Curve for Glucose

Same as mentioned in 3.1.3. (i).

(ii) Effect of Interfering Substances

Same as mentioned in 3.1.3. (ii).

3.5 Procedures or Methods for Employing Chitosan

3.5.1. Surface Cleaning and Generation of Hydroxyl Groups on GCE

Same as mentioned in 3.1.1.

3.5.2. Developed Highly-Simplified Chitosan Based Bienzyme Strategy

2 μL of 0.1 mg mL⁻¹ chitosan (CS) dispersed in 0.5% APTES was drop-casted on GCE followed by immediate drop-casting of 2 μL of bienzyme solution 1 to form CS-GOx-HRP mixture on GCE. The CS-GOx-HRP/GCE was dried at RT for 1 h and then washed extensively with 50 mM PBS. Thereafter, it was drop-casted with 3 μL of 0.5% Nafion and dried at RT for 10 min to form Nafion/CS-GOx-HRP/GCE followed by extensive washing with 50 mM PBS.

3.5.3. Electrochemical Analysis

Same as mentioned in 3.1.3.

(i) Assay Curve for Glucose

Same as mentioned in 3.1.3. (i).

(ii) Effect of Interfering Substances

Same as mentioned in 3.1.3. (ii).

The devices, structures, and techniques described herein are applicable to various electrode materials such as platinum, gold, carbon, glassy carbon, and many other substrates; and are suitable for use with a wide variety of immobilization agents, including nano-scale species, structures, or materials such as graphene, multi-walled carbon nanotubes, nanocrystalline cellulose, chitosan, poly-l-lysine, nanoparticles, polymers, nanocomposites, etc. Various types of biomolecules can be immobilized or bound in accordance with the teachings herein, such as enzymes, proteins, concanavalin A (glucose binding protein), and/or other biomolecules.

While various aspects and embodiments have been disclosed herein, it will be apparent that various other modifications and adaptations of the invention will be apparent to the person skilled in the art after reading the foregoing disclosure without departing from the spirit and scope of the invention and it is intended that all such modifications and adaptations come within the scope of the appended claims. The various aspects and embodiments disclosed herein are for purposes of illustration and are not intended to be limiting, with the true scope and spirit of the invention being indicated by the appended claims.

### REFERENCES

[1] Analytica Chimica Acta; 2011; DOI: 10.1016/j.aca.2011.07.024. Technology behind commercial devices for blood glucose monitoring in diabetic management: A review

1. A mediator-less biosensor for detecting an analyte within a detection environment, the mediator-less biosensor comprising:
a substrate having an electrically conductive chemically modified surface to which a functionalizing agent is covalently bonded; and
a first enzyme immobilized relative to the surface by way of covalent bonding to one of the functionalizing agent, a polymer chemically bonded to the functionalizing agent, and a nano-engineered material chemically bonded to the functionalizing agent, wherein the first enzyme is suitable for detecting one of glucose, cholesterol, alcohol, lactate, acetylcholine, choline, hypoxanthine, and xanthine,
wherein the mediator-less biosensor is configured for direct electron transfer between the analyte and the first enzyme in response to application of a negative electrical potential to the surface relative to the detection environment,
wherein the first enzyme becomes covalently bonded to the surface by way of (a) exposure of the surface to a fluid medium carrying a mixture of the first enzyme and the functionalizing agent, (b) exposure of the surface to a suspension comprising the polymer and the functionalizing agent, or (c) exposure of the surface to a fluid medium comprising the functionalizing agent, the polymer, and the first enzyme,
wherein the nano-engineered material includes at least one of graphene nano-platelets, multi-walled carbon nanotubes, and nanocrystalline cellulose, and
wherein the substrate carries one of a metal and a carbon based material.

2. (canceled)

3. The mediator-less biosensor of claim 1, wherein the electrically conductive chemically modified surface carries hydroxyl groups to which the functionalizing agent is covalently bound, and/or wherein the functionalizing agent comprises an organofunctional alkoxysilane compound.

4-8. (canceled)

9. The mediator-less biosensor of claim 1, wherein the polymer comprises one of an amino acid polymer and a glucosamine based polymer.

10-16. (canceled)

17. The mediator-less biosensor of claim 1, wherein the mediator-less biosensor is capable of detecting glucose across substantially the entire diabetic pathological concentration range, including a glucose concentration range of approximately 0.5-32 mM.

18-22. (canceled)

23. The mediator-less biosensor of claim 1, wherein the nano-engineered material becomes covalently bonded to the surface by way of exposure of the surface to (a) the functionalizing agent thereby creating a functionalized surface, followed by exposure of the functionalized surface to a polar dispersion agent carrying the nano-engineered material, (b) a suspension comprising the nano-engineered material and the functionalizing agent, or (c) a fluid medium comprising the functionalizing agent, the nano-engineered material, and the first enzyme.

24-25. (canceled)

26. The mediator-less biosensor of claim 1, further comprising a second enzyme immobilized relative to the surface by way of covalent bonding to one of the functionalizing agent, the polymer, and the nano-engineered material, wherein the second enzyme can reduce a byproduct of an electrochemical analyte detection reaction, and/or the second enzyme increases at least one of dynamic analyte detection range and analyte detection sensitivity.

27-29. (canceled)

30. The mediator-less biosensor of claim 26, wherein the first enzyme comprises glucose oxidase, and wherein the mediator-less biosensor is capable of detecting glucose across a concentration range of approximately 0.5-48 mM.

31. The mediator-less biosensor of claim 26, wherein the first enzyme and the second enzyme become covalently bonded to (a) the functionalizing agent by way of exposure of the surface to a fluid medium carrying the functionalizing agent, the first enzyme, and the second enzyme, (b) the polymer by way of exposure of the surface to a fluid medium carrying the functionalizing agent, the polymer, the first enzyme, and the second enzyme, or (c) the nano-engineered material by way of exposure of the surface to a fluid medium carrying the functionalizing agent, the nano-engineered material, the first enzyme, and the second enzyme.

32-33. (canceled)

34. A method for manufacturing a mediator-less biosensor configured for detecting an analyte in a detection environment, the method comprising:
providing a substrate having an electrically conductive chemically modified surface by way of exposing an electrically conductive portion of the surface to a surface modification agent such that the surface carries hydroxyl groups;
covalently bonding a functionalizing agent to the surface; and
performing an immobilization process comprising one of:
(a) covalently bonding a first enzyme to the functionalizing agent by way of (i) exposure of the surface to the functionalizing agent thereby creating a functionalized surface, followed by exposure of the functionalized surface to the first enzyme, or (ii) exposure of the surface to a fluid medium carrying a mixture of the first enzyme and the functionalizing agent;
(b) covalently bonding a polymer to the functionalizing agent and covalently bonding the first enzyme to the polymer by way of (i) exposure of the surface to a suspension comprising the polymer and the functionalizing agent, or (ii) exposure of the surface to a fluid medium comprising the functionalizing agent, the polymer, and the first enzyme; and
(c) covalently bonding a nano-engineered material to the functionalizing agent and covalently bonding the first enzyme to the nano-engineered material by way of (i) exposure of the surface to a suspension comprising the nano-engineered material and the functionalizing agent, or (ii) exposure of the surface to a fluid medium comprising the functionalizing agent, the nano-engineered material, and the first enzyme,
wherein the mediator-less biosensor is configured for direct electron transfer between the analyte and the first enzyme in response to application of a negative electrical potential to the surface relative to the detection environment,
wherein the first enzyme is suitable for detecting one of glucose, cholesterol, alcohol, lactate, acetylcholine, choline, hypoxanthine, and xanthine,
wherein the nano-engineered material includes at least one of graphene nano-platelets, multi-walled carbon nanotubes, and nanocrystalline cellulose, and
wherein the substrate carries one of a metal and a carbon based material.

35-40. (canceled)

41. The method of claim 34, wherein the polymer comprises one of an amino acid polymer and a glucosamine based polymer.

42-48. (canceled)

49. The method of claim 34, wherein the mediator-less biosensor is capable of detecting glucose across substantially the entire diabetic pathological concentration range, including a glucose concentration range of approximately 0.5-32 mM.

50-57. (canceled)

58. The method of claim 34, wherein the immobilization process involves the first enzyme and a second enzyme different than the first enzyme, and wherein the immobilization process comprises one of:
(a) covalently bonding the first enzyme and the second enzyme to the functionalizing agent;
(b) covalently bonding a polymer to the functionalizing agent and covalently bonding the first enzyme and the second enzyme to the polymer; and
(c) covalently bonding a nano-engineered material to the functionalizing agent and covalently bonding the first enzyme and the second enzyme to the nano-engineered material.

59. The method of claim 58, wherein the second enzyme can reduce a byproduct of an electrochemical analyte detection reaction, and/or wherein the second enzyme increases at least one of dynamic analyte detection range and analyte detection sensitivity.

60-61. (canceled)

62. The method of claim 58, wherein the first enzyme comprises glucose oxidase, and wherein the mediator-less biosensor is capable of detecting glucose across a concentration range of approximately 0.5-48 mM.

63. The method of claim 58, wherein the first enzyme and the second enzyme become covalently bonded to (a) the functionalizing agent by way of exposure of the surface to a fluid medium carrying the functionalizing agent, the first enzyme, and the second enzyme, or (b) the polymer by way of exposure of the surface to a fluid medium carrying the functionalizing agent, the polymer, the first enzyme, and the second enzyme, or (c) the nano-engineered material by way of exposure of the surface to a fluid medium carrying the functionalizing agent, the nano-engineered material, the first enzyme, and the second enzyme.

64-75. (canceled)
